SEQUENCE LISTING (134 sequences)

SEQ ID NO: 1 - DNA, 20 bp, Artificial Sequence
  Synthetic oligonucleotide
ttcctccattctccctcctc

SEQ ID NO: 2 - DNA, 20 bp, Artificial Sequence
  Synthetic oligonucleotide
cctgggatagctaggggttc

SEQ ID NO: 3 - DNA, 20 bp, Artificial Sequence
  Synthetic oligonucleotide
cgtgaaccacctggacatag

SEQ ID NO: 4 - DNA, 20 bp, Artificial Sequence
  Synthetic oligonucleotide
ccagggaggcaattaggaac

SEQ ID NO: 5 - DNA, 20 bp, Artificial Sequence
  Synthetic oligonucleotide
agacgtgggttcggtatcag

SEQ ID NO: 6 - DNA, 20 bp, Artificial Sequence
  Synthetic oligonucleotide
catccttgtgccgcttgtag

SEQ ID NO: 7 - DNA, 23 bp, Artificial Sequence
  Synthetic oligonucleotide
tccttggtcaggcagtataatcc

SEQ ID NO: 8 - DNA, 24 bp, Artificial Sequence
  Synthetic oligonucleotide
gtcaagggcatatcctacaacaaa

SEQ ID NO: 9 - DNA, 21 bp, Artificial Sequence
  Synthetic oligonucleotide
cagacttccgctcgtacctaa

SEQ ID NO: 10 - DNA, 19 bp, Artificial Sequence
  Synthetic oligonucleotide
tcgccctggggtaagatgt

SEQ ID NO: 11 - DNA, 21 bp, Artificial Sequence
  Synthetic oligonucleotide
gatgctcagtagtcaagtgcc

SEQ ID NO: 12 - DNA, 22 bp, Artificial Sequence
  Synthetic oligonucleotide
gccgttacccactcactaatac

SEQ ID NO: 13 - DNA, 20 bp, Artificial Sequence
  Synthetic oligonucleotide
ccacatcaccctactccgtc

SEQ ID NO: 14 - DNA, 21 bp, Artificial Sequence
  Synthetic oligonucleotide
ttgcagaccttgttgtgacac

SEQ ID NO: 15 - DNA, 21 bp, Artificial Sequence
  Synthetic oligonucleotide
gcgtttgcgaagatgagaact

SEQ ID NO: 16 - DNA, 23 bp, Artificial Sequence
  Synthetic oligonucleotide
tggtgttacattcacatttccca

SEQ ID NO: 17 - DNA, 20 bp, Artificial Sequence
  Synthetic oligonucleotide
tgggtgcctgctatgacaag

SEQ ID NO: 18 - DNA, 20 bp, Artificial Sequence
  Synthetic oligonucleotide
ggctcggacactttcctgag

SEQ ID NO: 19 - DNA, 20 bp, Artificial Sequence
  Synthetic oligonucleotide
gctctcatcggccaatcact

SEQ ID NO: 20 - DNA, 19 bp, Artificial Sequence
  Synthetic oligonucleotide
tctatggcctgctccacgt

SEQ ID NO: 21 - DNA, 20 bp, Artificial Sequence
  Synthetic oligonucleotide
cgagacctccacacttcgtg

SEQ ID NO: 22 - DNA, 23 bp, Artificial Sequence
  Synthetic oligonucleotide
tgtttttccgcctcatttctctg

SEQ ID NO: 23 - DNA, 21 bp, Artificial Sequence
  Synthetic oligonucleotide
ctgaagcccaatatccccgaa

SEQ ID NO: 24 - DNA, 21 bp, Artificial Sequence
  Synthetic oligonucleotide
gtagcaccagtacgtggtctc

SEQ ID NO: 25 - DNA, 19 bp, Artificial Sequence
  Synthetic oligonucleotide
actggctcgggaagatgct

SEQ ID NO: 26 - DNA, 20 bp, Artificial Sequence
  Synthetic oligonucleotide
ccgatggatcactttggtcc

SEQ ID NO: 27 - DNA, 20 bp, Artificial Sequence
  Synthetic oligonucleotide
tggggtcactgcaccaaaat

SEQ ID NO: 28 - DNA, 21 bp, Artificial Sequence
  Synthetic oligonucleotide
gaaggtcggcttgcacacata

SEQ ID NO: 29 - DNA, 20 bp, Artificial Sequence
  Synthetic oligonucleotide
agctcctaccaccagtacgg

SEQ ID NO: 30 - DNA, 22 bp, Artificial Sequence
  Synthetic oligonucleotide
gtttgccattcaccattctcac

SEQ ID NO: 31 - DNA, 22 bp, Artificial Sequence
  Synthetic oligonucleotide
ttttacgggagccctatacagt

SEQ ID NO: 32 - DNA, 22 bp, Artificial Sequence
  Synthetic oligonucleotide
gcaacattgcctctcatggttt

SEQ ID NO: 33 - DNA, 21 bp, Artificial Sequence
  Synthetic oligonucleotide
ctggatgacccggcaaattct

SEQ ID NO: 34 - DNA, 22 bp, Artificial Sequence
  Synthetic oligonucleotide
cctcttcaggctcaatcagttt

SEQ ID NO: 35 - DNA, 22 bp, Artificial Sequence
  Synthetic oligonucleotide
tttctaaagaacgcgaacgtct

SEQ ID NO: 36 - DNA, 23 bp, Artificial Sequence
  Synthetic oligonucleotide
gcaatatgcacttacaggtttgg

SEQ ID NO: 37 - DNA, 23 bp, Artificial Sequence
  Synthetic oligonucleotide
catgagaagtatgacaacagcct

SEQ ID NO: 38 - DNA, 22 bp, Artificial Sequence
  Synthetic oligonucleotide
agtccttccacgataccaaagt

SEQ ID NO: 39 - DNA, 20 bp, Artificial Sequence
  Synthetic oligonucleotide
aggaacccctcggacaagtt

SEQ ID NO: 40 - DNA, 19 bp, Artificial Sequence
  Synthetic oligonucleotide
ccgcactctcgtagttgtg

SEQ ID NO: 41 - DNA, 19 bp, Artificial Sequence
  Synthetic oligonucleotide
gggctgggacatgcagaat

SEQ ID NO: 42 - DNA, 22 bp, Artificial Sequence
  Synthetic oligonucleotide
cgtctttggaacagtagagcaa

SEQ ID NO: 43 - DNA, 21 bp, Artificial Sequence
  Synthetic oligonucleotide
gtagccatgaatgagaccgac

SEQ ID NO: 44 - DNA, 20 bp, Artificial Sequence
  Synthetic oligonucleotide
ggatcggggtgagagtctgt

SEQ ID NO: 45 - DNA, 19 bp, Artificial Sequence
  Synthetic oligonucleotide
gacgccctcctatttgccg

SEQ ID NO: 46 - DNA, 19 bp, Artificial Sequence
  Synthetic oligonucleotide
ccacggtatggcacacact

SEQ ID NO: 47 - DNA, 20 bp, Artificial Sequence
  Synthetic oligonucleotide
tcctcgacagttgctccaag

SEQ ID NO: 48 - DNA, 20 bp, Artificial Sequence
  Synthetic oligonucleotide
gcagatgtggcgtgatccat

SEQ ID NO: 49 - DNA, 21 bp, Artificial Sequence
  Synthetic oligonucleotide
gtggggcacttaatggggaaa

SEQ ID NO: 50 - DNA, 23 bp, Artificial Sequence
  Synthetic oligonucleotide
ctatgagacccagcaaattcctt

SEQ ID NO: 51 - DNA, 20 bp, Artificial Sequence
  Synthetic oligonucleotide
ggtgaccaccccaacaacag

SEQ ID NO: 52 - DNA, 22 bp, Artificial Sequence
  Synthetic oligonucleotide
gaattttggcgaagtgcttctg

SEQ ID NO: 53 - DNA, 19 bp, Artificial Sequence
  Synthetic oligonucleotide
cctggtctactgccgcttg

SEQ ID NO: 54 - DNA, 20 bp, Artificial Sequence
  Synthetic oligonucleotide
gttgaagtgtgcggggtact

SEQ ID NO: 55 - DNA, 20 bp, Artificial Sequence
  Synthetic oligonucleotide
aacgggacaagaaactgcac

SEQ ID NO: 56 - DNA, 21 bp, Artificial Sequence
  Synthetic oligonucleotide
ctagcggttccttcatggact

SEQ ID NO: 57 - DNA, 22 bp, Artificial Sequence
  Synthetic oligonucleotide
tacaaaccagttgacctgagca

SEQ ID NO: 58 - DNA, 21 bp, Artificial Sequence
  Synthetic oligonucleotide
atggatgttgcctaatgagcc

SEQ ID NO: 59 - DNA, 23 bp, Artificial Sequence
  Synthetic oligonucleotide
atgccatcagtgaatcaaaggat

SEQ ID NO: 60 - DNA, 23 bp, Artificial Sequence
  Synthetic oligonucleotide
ctggtaaagtaggagttgctacg

SEQ ID NO: 61 - DNA, 21 bp, Artificial Sequence
  Synthetic oligonucleotide
ccagcgatctttttggaggtg

SEQ ID NO: 62 - DNA, 21 bp, Artificial Sequence
  Synthetic oligonucleotide
tggtgatggactgagaagagg

SEQ ID NO: 63 - DNA, 21 bp, Artificial Sequence
  Synthetic oligonucleotide
acatcacctgctacttcctga

SEQ ID NO: 64 - DNA, 23 bp, Artificial Sequence
  Synthetic oligonucleotide
cttggactcatctttcgagaagg

SEQ ID NO: 65 - DNA, 23 bp, Artificial Sequence
  Synthetic oligonucleotide
ctgagcatgtgcaacaattctac

SEQ ID NO: 66 - DNA, 22 bp, Artificial Sequence
  Synthetic oligonucleotide
acagctccaaaaaggctgattc

SEQ ID NO: 67 - DNA, 21 bp, Artificial Sequence
  Synthetic oligonucleotide
ggagttggatgggactgagaa

SEQ ID NO: 68 - DNA, 21 bp, Artificial Sequence
  Synthetic oligonucleotide
ctgagcaatgtggcgatacag

SEQ ID NO: 69 - DNA, 21 bp, Artificial Sequence
  Synthetic oligonucleotide
cagagacgctacaactcaccg

SEQ ID NO: 70 - DNA, 19 bp, Artificial Sequence
  Synthetic oligonucleotide
ccgtgatgttctgttcgcc

SEQ ID NO: 71 - DNA, 19 bp, Artificial Sequence
  Synthetic oligonucleotide
tgcaagcccttccactacg

SEQ ID NO: 72 - DNA, 20 bp, Artificial Sequence
  Synthetic oligonucleotide
tgttgtggcgacagttgagg

SEQ ID NO: 73 - DNA, 21 bp, Artificial Sequence
  Synthetic oligonucleotide
ctgacggtgcctatgctgttc

SEQ ID NO: 74 - DNA, 22 bp, Artificial Sequence
  Synthetic oligonucleotide
gaaggtgttgacctgtatgacg

SEQ ID NO: 75 - DNA, 23 bp, Artificial Sequence
  Synthetic oligonucleotide
ctctcatcatgcctatgcctact

SEQ ID NO: 76 - DNA, 21 bp, Artificial Sequence
  Synthetic oligonucleotide
cctccacctcctgtaaccaag

SEQ ID NO: 77 - DNA, 23 bp, Artificial Sequence
  Synthetic oligonucleotide
gggagattgataactggtgtgtt

SEQ ID NO: 78 - DNA, 23 bp, Artificial Sequence
  Synthetic oligonucleotide
gtgtatatcccagggtgatcctc

SEQ ID NO: 79 - DNA, 21 bp, Artificial Sequence
  Synthetic oligonucleotide
taggcaggggcataaggaaat

SEQ ID NO: 80 - DNA, 23 bp, Artificial Sequence
  Synthetic oligonucleotide
ctcaaaccaaggaatagcttcca

SEQ ID NO: 81 - DNA, 21 bp, Artificial Sequence
  Synthetic oligonucleotide
cagacactttcaggtcagagc

SEQ ID NO: 82 - DNA, 23 bp, Artificial Sequence
  Synthetic oligonucleotide
aggcatcatactagacttgtgct

SEQ ID NO: 83 - DNA, 22 bp, Artificial Sequence
  Synthetic oligonucleotide
agaggccagcagaaaagtgact

SEQ ID NO: 84 - DNA, 21 bp, Artificial Sequence
  Synthetic oligonucleotide
gtggctcagcagattgaagaa

SEQ ID NO: 85 - DNA, 19 bp, Artificial Sequence
  Synthetic oligonucleotide
ccaagtacgcggacctgtc

SEQ ID NO: 86 - DNA, 20 bp, Artificial Sequence
  Synthetic oligonucleotide
ctcgcacgttagactctgga

SEQ ID NO: 87 - DNA, 21 bp, Artificial Sequence
  Synthetic oligonucleotide
catggagatggcctgtgacta

SEQ ID NO: 88 - DNA, 19 bp, Artificial Sequence
  Synthetic oligonucleotide
ggaagggcggaaactggtc

SEQ ID NO: 89 - DNA, 21 bp, Artificial Sequence
  Synthetic oligonucleotide
agtctgctgggcaaaagacaa

SEQ ID NO: 90 - DNA, 20 bp, Artificial Sequence
  Synthetic oligonucleotide
aggctcacttagtgctgggt

SEQ ID NO: 91 - DNA, 21 bp, Artificial Sequence
  Synthetic oligonucleotide
ggcctgggattcagtattgct

SEQ ID NO: 92 - DNA, 23 bp, Artificial Sequence
  Synthetic oligonucleotide
cccgcaagatacaatcattgacc

SEQ ID NO: 93 - DNA, 22 bp, Artificial Sequence
  Synthetic oligonucleotide
tcacaacctcttattcccagca

SEQ ID NO: 94 - DNA, 19 bp, Artificial Sequence
  Synthetic oligonucleotide
catggctgtggggtagtcg

SEQ ID NO: 95 - DNA, 19 bp, Artificial Sequence
  Synthetic oligonucleotide
ccgtggggaagacatgctt

SEQ ID NO: 96 - DNA, 21 bp, Artificial Sequence
  Synthetic oligonucleotide
accatcacgttggcagagtag

SEQ ID NO: 97 - DNA, 21 bp, Artificial Sequence
  Synthetic oligonucleotide
tctccccctcagttatgtgac

SEQ ID NO: 98 - DNA, 22 bp, Artificial Sequence
  Synthetic oligonucleotide
aggcaagtcttccaactttgaa

SEQ ID NO: 99 - DNA, 20 bp, Artificial Sequence
  Synthetic oligonucleotide
tggggagtggatagagacca

SEQ ID NO: 100 - DNA, 23 bp, Artificial Sequence
  Synthetic oligonucleotide
gaaagagattcaggaccactagg

SEQ ID NO: 101 - DNA, 23 bp, Artificial Sequence
  Synthetic oligonucleotide
ggtgattggaaatctagtggttc

SEQ ID NO: 102 - DNA, 20 bp, Artificial Sequence
  Synthetic oligonucleotide
catccttcccacagcaatct

SEQ ID NO: 103 - DNA, 20 bp, Artificial Sequence
  Synthetic oligonucleotide
agtagtggtgcattggcctt

SEQ ID NO: 104 - DNA, 20 bp, Artificial Sequence
  Synthetic oligonucleotide
gcaacccatttgagaagcat

SEQ ID NO: 105 - DNA, 18 bp, Artificial Sequence
  Synthetic oligonucleotide
acaggaagaggcacaggc

SEQ ID NO: 106 - DNA, 20 bp, Artificial Sequence
  Synthetic oligonucleotide
cccctccttcttcaccttct

SEQ ID NO: 107 - DNA, 23 bp, Artificial Sequence
  Synthetic oligonucleotide
attgtttgctggactctacagtg

SEQ ID NO: 108 - DNA, 21 bp, Artificial Sequence
  Synthetic oligonucleotide
ctttcaacagacgctcatcgt

SEQ ID NO: 109 - DNA, 19 bp, Artificial Sequence
  Synthetic oligonucleotide
gctgctgtctgaacccaac

SEQ ID NO: 110 - DNA, 19 bp, Artificial Sequence
  Synthetic oligonucleotide
cgttctcggggtgccatag

SEQ ID NO: 111 - DNA, 20 bp, Artificial Sequence
  Synthetic oligonucleotide
cagagtggcgagtccttgtc

SEQ ID NO: 112 - DNA, 21 bp, Artificial Sequence
  Synthetic oligonucleotide
tttcttccggctcgtttttga

SEQ ID NO: 113 - DNA, 21 bp, Artificial Sequence
  Synthetic oligonucleotide
tgaagccggattttgtgctga

SEQ ID NO: 114 - DNA, 21 bp, Artificial Sequence
  Synthetic oligonucleotide
gaccaaactgcggtagtctcc

SEQ ID NO: 115 - DNA, 21 bp, Artificial Sequence
  Synthetic oligonucleotide
tggcagacgactgaagaagag

SEQ ID NO: 116 - DNA, 22 bp, Artificial Sequence
  Synthetic oligonucleotide
ggatttggtcaatgttctcggg

SEQ ID NO: 117 - DNA, 23 bp, Artificial Sequence
  Synthetic oligonucleotide
ttcatagcgagtggtactttggc

SEQ ID NO: 118 - DNA, 21 bp, Artificial Sequence
  Synthetic oligonucleotide
agtctgggtttcaagggatca

SEQ ID NO: 119 - DNA, 23 bp, Artificial Sequence
  Synthetic oligonucleotide
cattatctcgaccactgacaacc

SEQ ID NO: 120 - DNA, 19 bp, Artificial Sequence
  Synthetic oligonucleotide
agaccccgtccaagacagg

SEQ ID NO: 121 - DNA, 21 bp, Artificial Sequence
  Synthetic oligonucleotide
gccctaccaagaccagacgta

SEQ ID NO: 122 - DNA, 19 bp, Artificial Sequence
  Synthetic oligonucleotide
cgtgaggcatagctcctga

SEQ ID NO: 123 - DNA, 22 bp, Artificial Sequence
  Synthetic oligonucleotide
cagagcactacaaacaccactg

SEQ ID NO: 124 - DNA, 19 bp, Artificial Sequence
  Synthetic oligonucleotide
ctgagtccgatctggctgg

SEQ ID NO: 125 - DNA, 21 bp, Artificial Sequence
  Synthetic oligonucleotide
aagttcactatcccggagtcc

SEQ ID NO: 126 - DNA, 23 bp, Artificial Sequence
  Synthetic oligonucleotide
tctgtcttttcacttgccagttt

SEQ ID NO: 127 - DNA, 21 bp, Artificial Sequence
  Synthetic oligonucleotide
acaagcaggcagagattgtca

SEQ ID NO: 128 - DNA, 21 bp, Artificial Sequence
  Synthetic oligonucleotide
tccatgtgataaatgctgggc

SEQ ID NO: 129 - DNA, 21 bp, Artificial Sequence
  Synthetic oligonucleotide
ctgagacccgagcagagtttg

SEQ ID NO: 130 - DNA, 21 bp, Artificial Sequence
  Synthetic oligonucleotide
tgaatctcgacgttctcctcc

SEQ ID NO: 131 - DNA, 19 bp, Artificial Sequence
  Synthetic oligonucleotide
agaccaatgagacggcctg

SEQ ID NO: 132 - DNA, 22 bp, Artificial Sequence
  Synthetic oligonucleotide
cctcctctacgttgtaaagggt

SEQ ID NO: 133 - DNA, 21 bp, Artificial Sequence
  Synthetic oligonucleotide
tccaatctcccgatgtcaagt

SEQ ID NO: 134 - DNA, 20 bp, Artificial Sequence
  Synthetic oligonucleotide
tcggcactgaaaacgatgct

(19) United States
(12) Patent Application Publication
(10) Pub. No.: US 2012/0277111 A1
(43) Pub. Date: Nov. 1, 2012

(54) MicroRNA Mediated Neuronal Cell Induction

(76) Inventors: Gerald R. Crabtree, Woodside, CA (US); Andrew Yoo, St. Louis, MO (US)

(21) Appl. No.: 13/441,673

(22) Filed: Apr. 6, 2012

Related U.S. Application Data
(60) Provisional application No. 61/473,558, filed on Apr. 8, 2011.
(60) Provisional application No. 61/486,102, filed on May 13, 2011.

(51) Int. Cl. C12N 5/0793; C12Q 1/68; G01N 27/26; G01N 33/566; C12N 15/85; C40B 30/04

(52) U.S. Cl. 506/9; 435/377; 435/455; 435/368; 435/34; 435/7.21; 435/6.12

(57) ABSTRACT

Methods of converting non-neuronal somatic cells into induced neuronal cells are provided. Aspects of the methods include contacting a non-neuronal somatic cell with a microRNA mediated neuronal cell induction agent. Aspects of the invention further include compositions produced by methods of the invention as well as compositions that find use in practicing embodiments of methods of invention. The methods and compositions find use in a variety of different applications.

### CROSS-REFERENCE TO RELATED APPLICATIONS

Pursuant to 35 U.S.C. §119 (e), this application claims priority to the filing date of U.S. Provisional Patent Application Ser. No. 61/473,558 filed on Apr. 8, 2011 and U.S. Provisional Patent Application Ser. No. 61/486,102 filed on May 13, 2011; the disclosures of which applications are herein incorporated by reference.

### GOVERNMENT RIGHTS

This invention was made with support under HD55391, A1060037 and NS046789 awarded by the National Institutes of Health. The Government has certain rights in the invention.

### INTRODUCTION

The diverse cell types present in the adult organism are produced during development by lineage-specific transcription factors that define and reinforce cell type specific gene expression patterns. Cellular phenotypes are further stabilized by epigenetic modifications that allow faithful transmission of cell-type specific gene expression patterns over the lifetime of an organism (Jenuwein, T. & Allis, C. D. (2001) Science 293, 1074-80; Bernstein, B. E., et al. (2007) Cell 128, 669-81). Recent work by Yamanaka and colleagues showing that four transcription factors are sufficient to induce pluripotency in primary fibroblasts demonstrated that fully differentiated cells can be induced to undergo dramatic cell fate changes (Takahashi, K. & Yamanaka, S. (206) Cell 126, 663-76). Similarly, the transfer of somatic cell nuclei into oocytes, as well as cell fusion of pluripotent cells with differentiated cells have proven to be capable of inducing pluripotency (Briggs, R. & King, T. J. (1952) Proc Natl Acad Sci USA 38, 455-63; Gurdon, J. B., et al. (1958) Nature 182, 64-5; Campbell, K. H., et al. (1996) Nature 380, 64-6; Tada, M., et al. (2001) Curr Biol 11, 1553-8; Do, J. T. & Scholer, H. R. (2004) Stem Cells 22, 941-9; Cowan, C. A., et al. (2005) Science 309, 1369-73). This transformation has been interpreted as a reversion of mature into more primitive developmental states, with a concomitant erasure of developmentally relevant epigenetic information (Silva, J. & Smith, A. (2008) Cell 132, 532-6). The resultant cells may then be reprogrammed to a new cell fate.

Reprogramming into an embryonic state with subsequent differentiation of the embryonic-state cells into cells of the Central Nervous System (CNS) is slow and inefficient, requiring significant time and manipulation in vitro. More useful would be direct reprogramming between divergent somatic lineages. It has been observed that cell fusion or forced expression of lineage-specific genes in somatic cells can induce traits of other cell types (Blau, H. M. (1989) Trends Genet 5, 268-72; Zhou, Q. & Melton, D. A. (2008) Cell Stem Cell 3, 382-8). For example, the basic helix-loop-helix (bHLH) transcription factor MyoD can induce muscle-specific properties in fibroblasts but not hepatocytes (Davis, R. L., et al. (1987) Cell 51, 987-1000; Schafer, B. W., et al. (1990) Nature 344, 454-8); ectopic expression of IL2 and GM-CSF receptors can lead to myeloid conversion in committed lymphoid progenitor cells (Kondo, M. et al. (2000) Nature 407, 383-6); expression of CEBPα in B-cells or Pu.1 and CEBPα in fibroblasts induces characteristics of macrophages (Bussmann, L. H. et al. (2009) Cell Stem Cell 5, 554-66; Feng, R. et al. (2008) Proc Natl Acad Sci U S A 105, 6057-62; Xie, H., et al. (2004) Cell 117, 663-76) deletion of PaxS can induce B-cells to de-differentiate toward a common lymphoid progenitor (Cobaleda, C., et al. (2007) Nature 449, 473-7); and the (bHLH) transcription factor neurogenin3, in combination with Pdx1 and MafA, can efficiently convert pancreatic exocrine cells into functional β-cells in vivo (Zhou, Q., et al. (2008) Nature 455, 627-32).

### SUMMARY

Methods of converting non-neuronal somatic cells into induced neuronal cells are provided. Aspects of the methods include contacting a non-neuronal somatic cell with a microRNA mediated neuronal cell induction agent. Aspects of the invention further include compositions produced by methods of the invention as well as compositions that find use in practicing embodiments of methods of invention. The methods and compositions find use in a variety of different applications.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1. MicroRNA-induced transformation of human fibroblasts. a, Morphological changes of fibroblasts induced by microRNAs. Neonatal foreskin fibroblasts infected with lentivirus to overexpress either miR-9/9*and -124 (miR-9/9*-124) or non-specific microRNA (miR-NS) are marked by RFP in order to monitor morphological changes. The photographs show the transition of the infected cells from the same well over the period of 20 days post-infection shown in columns. Two rows on top represent overexpression of miR-9/9*-124 without and with NeuroD2, respectively. The bottom two rows display cells expressing miR-NS in the absence or presence of NeuroD2. Scale bar=20 μm. b, Neuronal conversion of the infected fibroblasts. To assay for conversion of the infected fibroblasts towards neuronal cells, cells were immunostained with a MAP2 antibody. The photographs show MAP2-positive cells in green after four weeks post-infection by miR-9/9*-124 only or miR-9/9*-124-NeuroD2-overexpressing lentivirus. The graph represents scoring of converted fibroblasts by counting MAP2-positive cells with processes at least three times the cell body from 10 random fields. A total of 1558 and 658 cells were counted for miR-9/9*-124 only and miR-9/9*-124-NeuroD2, respectively. The error bars are in S.E.M. We did not detect any MAP2-positive cell in fibroblasts infected with miR-NS with or without NeuroD2. Scale bar=10 μm. c, Expression of other neuronal markers. The photographs show cells converted by miR-9/9*-124 and NeuroD2, immunostained with b-Ill tubulin and Synapsin1 shown in green. Scale bar=10 μm. d, Anti-proliferation effect of miR-9/9*-124 and NeuroD2. The proliferation of the infected cells was assayed by EdU (5-ethynyl-2′-deoxyuridine)-incorporation pulsed by 2-hour EdU treatment. The graph represents the percentage of EdU-positive cells out of total RFP-positive cells counted on day 8 post-infection. The percentages were averaged from 9-10 random fields of each condition. Note that NeuroD2 alone does not completely inhibit proliferation, consistent with our finding that NeuroD2 by itself is not sufficient for neuronal transformation. The error bars are S.E.M. e, Photographs show a representative converted cell immunostained for Scn1a, alpha subunit of type 1 voltage-gated sodium channel shown in green. The staining is localized to the initial segment of a process followed by punctate patterns of expression along the process. Scale bar=10 μm. f, An example of MAP2-positive converted cells expressing VGUT1 shown in green. Scale bar=10 μm. g, Immunostaining with an antibody against TBR1 shown in green in MAP2-positive converted cells. TBR1 staining is aligned with DAPI (in blue) to illustrate the nuclear localization of the staining. Scale bar=10 μm. h, Immunostaining with an antibody against R1 subunit of NMDA receptor shown in green in MAP2-positive converted cells. Positive staining is usually observed in the initial segment of a process and in punctate patterns along the processes. Scale bar=10 μm.

FIG. 2. Functional studies of the induced neurons. a, Representative traces of action potentials recorded in current clamp in miR-9/9*-124-NeuroD2-converted cells. Out of 16 cells that were recorded, 7 cells displayed single action potentials. b, A representative example of an induced neuron which was held at −70 mV and stepped from −70 mV to +70 mV in 10 mV increments. An inward current was observed, and was blocked by 1 μM TTX. The block is reversed after washing out of TTX (N=6). c, I-V curve for the peak inward (left) and outward (right) currents.d, An example of Ca²⁺ influx in induced neurons as measured by Fluo2-AM imaging. tRFP-positive cells indicate the infected cells expressing miR-9/9*-124 and NeuroD2 (top photo). The middle and bottom photos show the peak Fluo2-AM signal upon stimulation with or without TTX, respectively. The graph plots the changes in Fluo2 signal over time (circles, no TTX; triangles with TTX). Field stimulation is indicated by the black bar. All error bars represent standard error of the mean (±SEM). In some cases, the SEM is too small to be resolved. Scale bar=2 μm. e, An example of vesicle recycling measured by FM 1-43 imaging in induced neurons. Top diagram indicates the protocol of FM uptake or release experiments. The photos represent typical FM 1-43 dye uptake signal (left) in infected cell marked by tRFP (middle). The top graph represents quantification of FM 1-43 release (destaining) during stimulation. The bottom graph shows 0.1 mM Ca²⁺ significantly reduced uptake of FM types into synaptic vesicles. The graphs illustrate quantification of FM 1-43 signal from more than 600 boutons (exemplified by the arrows in the picture) from 4 cultures. Scale bar=2 μm.

FIG. 3. Electrophysiological properties of miR-9/9*-124-DAM cells. a, Representative current clamp recordings from the cell with a typical neuronal morphology (picture above). Voltage deflections were elicited by somatic current injections of various amplitude (Δ=10 pA). 23 out of 24 cells responded with action potentials and 19 of them showed repetitive firing. b, Representatives voltage clamp recordings of the net current at various membrane potentials (−80−+20, ΔV=10 mV, Vhold=−90 mV) c, A representative trace of spontaneous synaptic activities, measured at −70 mV d, Immunostaining of miR-9/9*-124-DAM-converted cells for MAP2. Scale bar=20 μm. The graph represents the percentage of MAP2-positive cells in a total number of DAPI-positive cells in a given field. Total number of cells counted=150 cells. e, Immunostaining of miR-9/9*-124-DAM-converted cells for 13111 tubulin. Scale bar=20 μm.

FIG. 4. Expression of subunits of neuron-specific BAF complexes. Photographs show fibroblasts and converted neurons stained for BAF45b in a, BAF45c in b, and BAF53b in c, shown in red. Top panels show fibroblasts expressing no or low amount of the neuron-specific subunits of BAF complexes. The bottom three panels show upregulated expression of the neuron-specific subunits (shown in red) in MAP2-positive (shown in green) cells. Scale bar=10 μm.

FIG. 5. Relative amount of microRNAs expressed in human fibroblasts in comparison to human brain. Quantitative real time PCR was performed from human fibroblasts expressing non-specific microRNA (miR-NS) or the synthetic cluster of mir-9/9*-124) to estimate how much miR-9/9* and miR-124 are expressed compared to the level found in human brains.

FIG. 6. Immunostaining for progenitor markers during the time course of conversion. The top left panel shows the positive control for Pax6 antibody staining (mouse neural progenitors). The top right photo shows human fibroblast stained for Pax6. The bottom pictures shows human fibroblasts expressing NeuroD2, miR-9.9*-124 and miR-9/9*-124-NeuroD2 stained for Pax6. We sampled every three days for 3 weeks. Here we show examples of the cells sampled on Day 3, 9 and 15. We did not observed any expression of Pax6 during the time course of the conversion. Similar results were obtained for Sox2 and Tbr2. Note that no MAP2-positive cells expressing only NeuroD2 were observed throughout the entire time course. Scale bar=20 um

FIG. 7. Immunostaining for keratinocytes in the starting culture of human fibroblasts. a) Top pictures show the starting culture of fibroblasts stained with Fibronectin and Vimentin. We observed homogenous population of fibroblasts characterized by high expression of Fibronectin and Vimentin. b) The starting culture of fibroblasts were stained with thee different markers for keratinocytes including K5, K14 and p63. The top row shows positive control from human keratinocytes for K5, K14, and p63, which were not expressed in the starting culture of human fibroblasts, as illustrated in the bottom panels. Scale bar=20 um

FIG. 8. Immunostaining for melanocytes in the starting culture of human fibroblasts. The first column representes pictures ofhuman malnocytes stained with anitbodies against MelaninA, MITF and p75 as a positive control. The second and third columns represent the pictures from human neonatal fibroblasts and human adult fibroblasts also stained with MelanA, MITF and p75, respectively. We do not observe any melanocyte present in the fibroblasts cultures used in this study. Scale bar=20 um.

FIG. 9. Synergistic effect of miR-9/9* and miR-124 on transformation. Left photos show human fibroblasts expressing miR-9/9* only (top), miR-124 only (middle) and miR-9/9*-124 together (bottom) without NeuroD2. tRFP indicates microRNA-expressing cells. Right photos show microRNA-expressing cells with NeuroD2. MAP2-positive cells appear only when miR-9/9* and miR-124 are expressed together with or without NeuroD2, demonstrating the synergistic effect of miR-9/9* and -124 on neuronal transformation of human fibroblasts. Scale bar=20 um.

FIG. 10. Effect of neurogenic factors on miR-9/9*-124-mediated conversion of human fibroblasts. NGN1=neurogenin1, NGN2=Neurogenin2, ND1=NeuroD1, ND2=NeuroD2. Top photos show MAP2-positive cells with respective neural factors co-expressed with miR-9/9*-124. Total scored numbers of MAP12-positive cells are ASCL1: 7/180, NGN1: 6/76, NGN2: 1/84, ND1:6/57, ND2: 28/81. *:p<0.01 by Student T-test between ND2 and ASCL1, NGN1, NGN2 or ND1. Scale bar=20 um.

FIG. 11. Exemplary pictures of Edu-incorproation assays on day 8 post-infection. Edu-positive cells are shown in green in four conditions: miR-9/9*-124 overexpression (top left). miR-9/9*-124 with NeuroD2 overexpression (top right), non-specific microRNA, miR-NS overexpression (lower left), and miR-NS with NeuroD2 oeverexpression (lower right panel).

FIG. 12. Conversion of arrested cells. Human neonatal foreskin fibroblasts were treated with either 10 ug/ml mitomycin C (MMC) or vehicle (Control) for 3 hours. A) 3 hour-Edu-pulsing confirmed that MMC treatment effectively inhibited cell proliferation, as compared with control treatment. MMC- and control-treated fibroblasts were transduced to express miR-9/9*-124 and NeuroD2 24 hours later. B) Photographs in panel b were taken from MMC-treated cells. As indicated by bill tubulin and MAP2 expression 20 days post-infection, MMC-treated cells were transformed into neurons, demonstrating that miRNA-mediated neuronal conversion is direct, wthout going through cell divisions.

FIG. 13. Stable transformation of human fibroblasts using doxycycline (Dox) inducible promoter to express miR-9/9*-124. After 20 days of induction, Dox was either kept (Dox on) or removed (Dox off) and the cells were analyzed for MAP2 expression after 7 more days. The graph shows the percentage of MAP2-positive cells in the remaining culture. The removal of Dox did not cause the induced cells to revert to fibroblast states. Scale bar=20 um. NS=Not significant, Student T-Test, p=0.09.

FIG. 14. Table describing data of quantitative real time PCR for neuronal genes. RT-qPCR was performed to assay the upregulation of neuronal genes including MAP2, VGLUT1, and NMDAR1. HN=human neurons as a positive control, IN=induced neurons by miR-9/9*-124 and NeuroD2 (20 days post-infection), Fb=human fibroblast as a negative control. All the values were normalized to HPRT reference values. **For VGLUT1 and NMDAR1, respective primers amplified (Ct values are provided) the transcrips in human neurons and induced neurons, whereas in Fb sample the same primers never amplified (with Ct values higher than 40). Thus, ** denotes significant expressions in HN and IN samples compared to Fb samples.

FIG. 15. Summary of electrophysiological properties. Resting membrane potentials and capacitances of induced cells are summarized.

FIG. 16. Electrophysiological properties of fibroblasts expressing non-specific microRNA. Top diagram shows an exemplary voltage-clamp analysis of fibroblasts, showing the absence of inward current. The bottom diagram shows an exemplary current clamp analysis of fibroblasts. Note that fibroblasts do no generate inward currents.

FIG. 17. An example of Fluo2AM calcium imaging displaying action potential (AP)-dependent Ca2+ influx. The pictures show the increase in FLuo2 signal during stimulation, which was blocked by TTX. After TTX was washed away, the same cell was treated with CD2+ which also blocked the Ca2+ influx.

FIG. 18. Representative traces of action potentials displayed in miR-9/9*-124-DAM-induced neurons.

FIG. 19. Cells infected with non-specific microRNA (miR-NS) and DAM factors are stained by antibodies against MAP2 (top) and VGLUT1 (bottom). miR-NS-DAM treatment does not lead to induction of MAP2- and VGLU1-expressing neurons.

FIG. 20. Schematic representation of real time RT-PCR on single cels collected after electrophysiological recordings. Black boxes represent detected mRNA of genes. CTRL: internal solution negative control. The list of primers used in provide in the Examples.

FIG. 21. Western blot analysis of BAF53a expression in human fibroblasts. Lane 1 represents native human fibroblasts showing the detection of BAF53a indicating that BAF53a is expressed in fibroblasts. When BAF53a is additionally expressed in fibroblasts, the level is increased as shown in lane 2, demonstrating the specificity of the antibody. Lane 3 represents fibroblasts expressing miR-9/9*-124 leads to downregulation of BAF53a.

FIG. 22. Conversion of adult human dermal fibroblasts. a, The photographs are taken from live cells on day 12 (top) and 4 weeks (bottom) after infection. Whereas neonatal fibroblasts already adopted neuronal morphologies by day 12, adult fibroblasts still retained the morphologies of fibroblasts. The bottom panel shows morphological changes of adult fibroblasts towards neuronal shapes after 4 weeks. Scale bar=5 mm b, Adult fibroblasts are fixed after 5 weeks and stained for b-Ill tubulin, MAP2, Neurofilament and VGLUT1. Adult fibroblasts were transformed to neurons characteristic of glutamatergic neurons, similar to neonatal fibroblast-derived neurons. All the neuronal markers are shown in green. Scale bar=10 μm c, The diagram shows voltage-activated sodium conductance during current clamp recording of a cell resting at −60 mV with increasing pulses of positive current (20 pA steps). The inset shows enlarged top trace focusing on the action potential (length 70 ms, height 45 mv).

FIG. 23. A representative diagram of whole cell recordings of adult fibroblast-derived neurons displaying sodium and potassium currents during voltage-clamping. Human adult fibroblasts were converted by miR-9/9*-124 and NeuroD2 and recorded approximately 40 days post-infection.

FIG. 24. Improvied efficiency of the production of neurons. Human fetal foreskin fibroblasts were infected with viruses expressing miR9/124 with or without BclXL. The graph represents the percentage of MAP2-positive cells in a total number of DAPI-positive cells in a given field.

FIG. 25. Production of human neurons from glia. Human glial cells were infected with viruses expressing miR9/124 and BclXL. After 30 days about 35% of the cells were converted to neurons. Neurons are Map2 positive and GFAP negative.

FIG. 26. Development of methods to produce inhibitory neurons. Use of miR9*, miR124, Ascl and Mytl1 gave populations of neurons about 50% of which appear to be inhibitory neurons, as determined by reactivity with an anti-GABA antibody.

### DEFINITIONS

The terms “induced neuronal cell,” “iN cell” “induced neuron,” or “iN” encompass cells of the neuronal lineage i.e. mitotic neuronal progenitor cells and post-mitotic neuronal precursor cells and mature neurons, that arise from a non-neuronal cell by experimental manipulation. Induced neuronal cells express markers specific for cells of the neuronal lineage, e.g. Tau, Tuj1, MAP2, NeuN, and the like, and may have characteristics of functional neurons, that is, they may be able to be depolarized, i.e. propagate an action potential, and they may be able to make and maintain synapses with other neurons.

The term “somatic cell” encompasses any cell in an organism that cannot give rise to all types of cells in an organism, i.e. it is not pluripotent. In other words, somatic cells are cells that have differentiated sufficiently that they will not naturally generate cells of all three germ layers of the body, i.e. ectoderm, mesoderm and endoderm.

The term “pluripotent” or “pluripotency” refers to cells with the ability to give rise to progeny that can undergo differentiation, under appropriate conditions, into cell types that collectively exhibit characteristics associated with cell lineages from the three germ layers (endoderm, mesoderm, and ectoderm). A “stem cell” is a cell characterized by the ability of self-renewal through mitotic cell division and the potential to differentiate into a tissue or an organ. Among mammalian stem cells, embryonic and somatic stem cells may be distinguished. Pluripotent stem cells, which include embryonic stem cells, embryonic germ cells and induced pluripotent cells, can contribute to tissues of a prenatal, postnatal or adult organism.

The terms “primary cells”, “primary cell lines”, and “primary cultures” are used interchangeably herein to refer to cells and cell cultures that have been d erived from a subject and allowed to grow in vitro for a limited number of passages, i.e. splittings, of the culture. For example primary cultures are cultures that may have been passaged 0 times, 1 time, 2 times, 4 times, 5 times, 10 times, or 15 times, but not enough times go through the crisis stage. Typically, the primary cell lines of the present invention are maintained for fewer than 10 passages in vitro.

The terms “efficiency of reprogramming”, “reprogramming efficiency”, “efficiency of conversion”, or “conversion efficiency” are used interchangeably herein to refer to the ability of a culture of cells of one cell lineage to give rise to an induced cell of another cell lineage when contacted with a microRNA mediated neuronal cell induction agent of the invention. By “enhanced efficiency of reprogramming” or “enhanced efficiency of conversion” it is meant an enhanced ability of a culture of somatic cells to give rise to the induced neuronal cell when contacted with the reprogramming system relative to a culture of somatic cells that is not contacted with the reprogramming system, for example, an enhanced ability of a culture of cells to give rise to iN cells when contacted with a microRNA mediated neuronal cell induction agent relative to a culture of cells that is not contacted with the same agent. By enhanced, it is meant that the primary cells or primary cell cultures have an ability to give rise to the induced neuronal cells (e.g., iN cells) that is greater than the ability of a population that is not contacted with the induction agent, e.g., 150%, 200%, 300%, 400%, 600%, 800%, 1000%, or 2000% of the ability of the uncontacted population. In other words, the primary cells or primary cell cultures produce 1.5-fold or more, 2-fold or more, 3-fold or more, 4-fold or more, 6-fold or more, 8-fold or more, 10-fold or more, 20-fold or more, 30-fold or more, 50-fold or more, 100-fold or more, 200-fold or more the number of induced cells (e.g. iN cells) as the uncontacted population.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

The terms “individual,” “subject,” “host,” and “patient,” are used interchangeably herein and refer to any mammalian subject for whom diagnosis, treatment, or therapy is desired, particularly humans.

### DETAILED DESCRIPTION

Methods of converting non-neuronal somatic cells into induced neuronal cells are provided. Aspects of the methods include contacting a non-neuronal somatic cell with a microRNA-mediated neuronal cell induction agent. Aspects of the invention further include compositions produced by methods of the invention as well as compositions that find use in practicing embodiments of methods of invention. The methods and compositions find use in a variety of different applications.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As summarized above, embodiments of the invention include methods of inducing neuronal cells from non-neuronal somatic cells. Aspects of these methods include contacting a non-neuronal somatic cell (or collection of non-neuronal somatic cells, e.g., culture or a present in a tissue of an organism) with a microRNA-mediated neuronal cell induction agent, where the neuronal cell induction agent is sufficient to cause microRNA-mediated conversion of the non-neuronal somatic cell into an induced neuronal cell. The specific nature of the induction agent may vary greatly depending on the particular embodiment of the methods being practiced. Examples of different types of induction agents include, but are not limited to: nucleic acids (e.g., microRNA or expression cassettes that encode the same, where the expression cassettes may be present in a vector), expression inducers, polypeptides, small molecules, and combinations thereof, where examples of these types of agents are described in further detail below.

As stated above, the induction agent is one or more components that, upon contact with a non-neuronal somatic cell, is sufficient to cause induction of the cell into a neuronal cell. The induced neuronal cell into which the somatic cell is converted upon contact with the induction agent may vary, where induced neuronal cells are as defined above. Methods describe heren may be used to produce a variety of different types of neurons, including projection (e.g., exitatory and inhibitory) neurons, interneurons, etc. In some instances, the induced neuronal cell may be further characterized as sharing one or more phenotypic traits with a naturally occuring neuronal cell, such as but not limited to: Unipolar or pseudounipolar cells, Bipolar cells, Multipolar cells, Golgi I cells, Golgi II cells, Basket cells, Betz cells, Medium spiny neurons, Purkinje cells, Golgi I multipolar neurons, Pyramidal cells, Renshaw cells, Granule cells, anterior horn cells, etc.

In some instances, the microRNA-mediated conversion that is caused by the induction agent includes providing a level of two or more microRNAs in the cell that is sufficient to cause the cell to convert to an induced neuronal cell. In other words, contact of the cell with the agent results in a level or concentration of two or more microRNAs, such as two distinct microRNAs, which is sufficient (i.e., at a value that) to cause conversion of the cell into a neuronal cell. In some instances, the induction agent is one that causes the level of two or more microRNAs in a cell to be sufficient to cause the cell to convert to an induced neuronal cell. In one such embodiment, a first microRNA of interest is miR-9. The sequence of miR-9 is reported at http://www.mirbase.org. See also Yoo, A. S., Staahl, B. T., Chen, L., & Crabtree, G. R., MicroRNA-mediated switching of chromatin-remodelling complexes in neural development. Nature 460 (7255), 642-646 (2009) In one such embodiment, a second microRNA of interest is miR-9*. The sequence of miR-9* is reported at the website having a address in which “www.” is placed before “mirbase.org.” See also Yoo, A. S., Staahl, B. T., Chen, L., & Crabtree, G. R., MicroRNA-mediated switching of chromatin-remodelling complexes in neural development. Nature 460 (7255), 642-646 (2009). In one such embodiment, a third microRNA of interest is miR-124. The seqeuence miR-124 is reported at the website having a address in which “www.” is placed before “mirbase.org.” See also Yoo, A. S., Staahl, B. T., Chen, L., & Crabtree, G. R., MicroRNA-mediated switching of chromatin-remodelling complexes in neural development. Nature 460 (7255), 642-646 (2009) Accordingly, in somes instances, the agent is one that, upon contact with the non-neuronal somatic cell, causes a level of one or more of miR-9*, -9 and miR-124 to be present in the cell that is sufficent to cause the cell to convert to a neuronal cell. While the level that is acheived by a given agent may vary, the level may be 25% or more, such as 50% or more, including 75% or more (e.g., 90% or more) of that observed in neurons derived from brain tissue, e.g., as determined via any convenient protocol, such as RT-PCR.

The particular induction agent employed in a given method may vary so long as the induction agent provides for the desired level of the two or more microRNAs in the cell. In some instances, the cell is contacted with mature versions of the two or more microRNAs of interest under conditions sufficient for the cell to internalize the microRNAs. For example, the cell may be contacted with two or more microRNAs in the presence of a transfection agent. Transfection agents of interest include, but are not limited to: Xfect™ transfection reagent from Clontech Laboratories, Lipofectamine LTX transfection reagent from Life Technologies, Lipofectamine 2000 transfection reagent from Life Technologies, SiQuest transfection reagent from Mirus, Transit-siQuest transfection reagent, Transit-TKO transfection reagent, Transit-LTI transfection reagent, Transit-Jurkat transfection reagent, Transit-2020 transfection reagent; chloroquine, PEG, etc. The particular transfection conditions may vary and any convenient protocol may be employed, where suitable protocols are known in the art. Methods for contacting cells with nucleic acid vectors, such as electroporation, calcium chloride transfection, and lipofection, are well known in the art. Vectors that deliver nucleic acids in this manner are usually maintained episomally, e.g. as plasmids or minicircle DNAs.

Instead of contacting the non-neuronal somatic cell with mature forms of the microRNAs of interest, the cell may be contacted with a vector that includes an expression cassette encoding the microRNA of interest or a precursor thereof, e.g., a primary micro-RNA molecule that can be processed by the cellular machinery of the non-somatic target cell into a pre-microRNA and then utimately cleaved into the microRNA. Any convenient coding sequence may be employed. For miR-9* and miR-9, coding sequences of interest include, but are not limited to, sequences that encode precursors of miR-9* and miR-9 (where both mature microRNAs are generated from the same precursor), e.g., where examples of such coding sequences are reported in http://www.mirbase.org. For miR-124, coding sequences of interest include, but are not limited to, sequences that encode precursors of miR-124, e.g., where examples of such coding sequences are reported in http://www.mirbase.org. A given vector may include a single coding sequence or multiple repeats of the coding sequence, as desired.

Vectors used for providing microRNA expression cassettes to the subject cells may include suitable promoters for driving the expression, that is, transcriptional activation, of the encoding sequence of the expression cassette. This may include ubiquitously acting promoters, for example, the CMV-β-actin promoter, or inducible promoters, such as promoters that are active in particular cell populations or that respond to the presence of drugs such as tetracycline. By transcriptional activation, it is intended that transcription will be increased above basal levels in the target cell by 10-fold or more, by 100-fold or more, such as by 1000-fold or more. In addition, vectors used for providing the nucleic acids may include genes that must later be removed, e.g., using a recombinase system such as Cre/Lox, or the cells that express them destroyed, e.g., by including genes that allow selective toxicity such as herpesvirus TK, bcl-xs, etc

Alternatively, the expression cassette(s) may be provided to the subject cells via a virus. In other words, the cells are contacted with viral particles comprising the expression cassettes. Retroviruses, for example, lentiviruses, are particularly suitable to such methods. Commonly used retroviral vectors are “defective”, i.e. unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein to be incorporated into the capsid, this envelope protein determining the specificity of the viral particle for the cells. Envelope proteins are of at least three types, ecotropic, amphotropic and xenotropic. Retroviruses packaged with ecotropic envelope protein, e.g. MMLV, are capable of infecting most murine and rat cell types, and are generated by using ecotropic packaging cell lines such as BOSC23 (Pear et al. (1993) P.N.A.S. 90:8392-8396). Retroviruses bearing amphotropic envelope protein, e.g. 4070A (Danos et al, supra.), are capable of infecting most mammalian cell types, including human, dog and mouse, and are generated by using amphotropic packaging cell lines such as PA12 (Miller et al. (1985) Mol. Cell. Biol. 5:431-437); PA317 (Miller et al. (1986) Mol. Cell. Biol. 6:2895-2902); GRIP (Danos et al. (1988) PNAS 85:6460-6464). Retroviruses packaged with xenotropic envelope protein, e.g. AKR env, are capable of infecting most mammalian cell types, except murine cells. The appropriate packaging cell line may be used to ensure that the subject cells are targeted by the packaged viral particles. Suitable methods of introducing the retroviral vectors comprising expression cassettes into packaging cell lines and of collecting the viral particles that are generated by the packaging lines are well known in the art.

In those embodiments where the microRNA mediated induction is mediated by three different microRNAs, e.g., miR-9*, miR-9 and miR-124, a single vector may be employed to introduce the expression cassettes of interest or a separate vector may be employed for each expression cassette.

In some embodiments (e.g., for enhanced efficiency of conversion), the non-neuronal somatic cell is also contacted with an agent that results in a desired activity of a neurogenic factor. As such, some embodiments of the methods include providing one or more neurogenic factor activities in the cell that enhance conversion of the cell to an induced neuronal cell. The neurogenic factor(s) may vary, and in some instances the neurogenic factor is a transcription factor. Transcription factors of interest include, but are not limited to: NeuroD polypeptides, NeuroD1, NeuroD2, NeuroD4, NeuroD6, Myt1I or Ascl-1 and the like.

In some instances, the transcription factor is a NeuroD polypeptide. NeuroD (neurogenic differentiation) polypeptides are basic helix-loop-helix transcription factors of the neurogenic differentiation family of proteins. The terms “NeuroD gene product”, “NeuroD polypeptide”, and “NeuroD protein” are used interchangeably herein to refer to native sequence NeuroD polypeptides, NeuroD polypeptide variants, NeuroD polypeptide fragments and chimeric NeuroD polypeptides that can modulate transcription. Native sequence NeuroD polypeptides include the proteins NeuroD1 (GenBank Accession Nos. NM_{—}002500.2 and NP_{—}002491.2); NeuroD2 (GenBank Accession Nos. NM_{—}006160.3 and NP_{—}006151.3); NeuroD4 (GenBank Accession Nos. NM_{—}021191.2 and NP_{—}067014.2) and NeuroD6 (NM_{—}022728.2 and NP_{—}073565.2). NeuroD polypeptides, e.g. those that are at least 50%, 55%, 60%, 65%, 70%, 75%, 80%, 85%, 90%, 91%, 92%, 95%, 97%, 99%, or are 100% identical to the sequence provided in the GenBank Accession Nos. above find use as reprogramming factors in the present invention, as do nucleic acids encoding these polypeptides or their transcriptionally active domains and vectors comprising these nucleic acids. In certain embodiments, the NeuroD agent is a NeuroD2 agent.

In some instances, the transcription factor is an Ascl-1 polypeptide. Ascl1 (achaete-scute-like) polypeptides are basic helix-loop-helix transcription factors of the achaete-scute family, which activate transcription by binding to the E box (5′-CANNTG-3′). The terms “Ascl gene product”, “Ascl polypeptide”, and “Ascl protein” are used interchangeably herein to refer to native sequence Ascl polypeptides, Ascl polypeptide variants, Ascl polypeptide fragments and chimeric Ascl polypeptides that can modulate transcription. Native sequence Ascl polypeptides include the proteins Ascl1 (achaete-scute complex homolog 1 (Drosophila); ASH1; HASH1; MASH1; bHLHa46; GenBank Accession Nos. NM_{—}004316.3 and NP_{—}004307.2); Ascl2 (achaete-scute complex homolog 2 (Drosophila); ASH2; HASH2; MASH2; bHLHa45; GenBank Accession Nos. NM_{—}005170.2 and NP_{—}005161.1); Ascl3 (achaete-scute complex homolog 3 (Drosophila); SGN1; HASH3; bHLHa42; GenBank Accession Nos. NM_{—}020646.1 and NP_{—}065697.1); Ascl4 (achaete-scute complex homolog 4 (Drosophila); HASH4; bHLHa44; GenBank Accession Nos. NM_{—}203436.2 and NP_{—}982260.2; and AsclS (achaete-scute complex homolog 5 (Drosophila); bHLHa47; GenBank Accession Nos. XM_{—}001719321.2 and XP_{—}001719373.2). Ascl polypeptides, e.g. those that are at least 50%, 55%, 60%, 65%, 70%, 75%, 80%, 85%, 90%, 91%, 92%, 95%, 97%, 99%, or are 100% identical to the sequence provided in the GenBank Accession Nos. above find use as reprogramming factors in the present invention, as do nucleic acids encoding these polypeptides or their transcriptionally active domains and vectors comprising these nucleic acids. In certain embodiments, the Ascl agent is an Ascl1 agent.

In some instances, the transcription factor is a Myt polypeptide. Myt (myelin transcription factor) polypeptides are members of the Myt family of zinc-finger transcription factors. The terms “Myt gene product”, “Myt polypeptide”, and “Myt protein” are used interchangeably herein to refer to native sequence Myt1 polypeptides, Myt polypeptide variants, Myt polypeptide fragments and chimeric Myt polypeptides that can modulate transcription. Native sequence Myt1 polypeptides include the proteins Myt1 (Nzf2; Nztf2; and mKIAA0835; GenBank Accession Nos. NM_{—}008665.3 and NP_{—}032691.2); and Myt1 I (myelin transcription factor 1-like; NZF1; Neural zinc finger transcription factor 1; GenBank Accession Nos. NM_{—}015025.2 and NP_{—}055840.2). Myt polypeptides, e.g. those that are at least 50%, 55%, 60%, 65%, 70%, 75%, 80%, 85%, 90%, 91%, 92%, 95%, 97%, 99%, or are 100% identical to the sequence provided in the GenBank Accession Nos. above find use as reprogramming factors in the present invention, as do nucleic acids encoding these polypeptides or their transcriptionally active domains and vectors comprising these nucleic acids. In certain embodiments, the Myt agent is a Myt1 I agent.

Where desired, embodiments of the methods described herein may include use of a conversion enhancement agent. By conversion enhancement agent is meant an agent that enhances conversion of the initial cells to the product neuronal cells 5% or more, such as 10% or more, including 20% or more, e.g., 30% or more, 40% or more, 50% or more, 75% or more, as compared to a suitable control (e.g., an identical protocol but for the lack of use of a conversion enhancement agent). In some embodiments, the conversion enhancement agent is a cell death reducing agent. By cell death reducing agent is meant an agent that reduces the occurrence of cell death in a given cellular population, e.g., where the magnitude of the reduction in occurrence of cell death may be 5% or more, such as 10% or more, including 20% or more, e.g., 30% or more, 40% or more, 50% or more, 75% or more, as compared to a suitable control (e.g., an identical protocol but for the lack of use of a conversion enhancement agent). The cell death reduction agent may exert activity in a number of different ways. Modes of cell death recognized in the art include, but are not limited to, apoptosis (i.e. programmed cell death), necrosis and autophagy. Agents of interest that inhibit or reduce cell death (cell death reducing agents) include, but are not limited to: antiapoptotic, antinecrotic, or antiautophagic agents.

As with the induction agent, the nature of the conversion enhancement agent may vary. In some instances, the conversion enhancement agent is a polypeptide (e.g., protein) or nucleic acid encoding the same. Proteins of interest include, but are not limited to, proteins known to have antiapoptotic activity, such as members of the BCL-2 protein family or members of the IAP (Inhibitor of Apoptosis) family.

In some instances, the conversion enhancement agent is a BCL-2 family member, such as BclXL. The terms “BclXL,” “BclXL gene product,” “BclXL polypeptide” and “BclXL protein” are used interchangeably herein to refer to native sequence BCL-2 protein family polypeptides, BCL-2 protein family polypeptide variants, BCL-2 protein family polypeptide fragments and chimeric BCL-2 protein family polypeptides that can modulate apoptosis. Native sequence BCL-2 protein family polypeptides include the proteins BclXL (Genbank Accession Nos. NM_{—}001191.2, 138578.1, NP_{—}001182.1 and NP_{—}612815.1; Aliases include: Bcl-XL, BCL-XL/S, BCL2L, BCLX, BCLXL, BCLXS, Bcl-X, PPP1 R52, bcl-xL and bcl-xS); Bcl2 (Genbank Accession Nos. NM_{—}000633.2, NM_{—}000657.2, NP_{—}000624.2 and NP_{—}000648.2; Aliases include: BCLW, BCL-W, PPP1 R51 and BCL2-L-2); Mcl-1 (Genbank Accession Nos. NM_{—}001197320.1, NM_{—}021960.4, NM_{—}182763.2, NP_{—}001184249.1, NP_{—}068779.1 and NP_{—}877495.1; Aliases include: BCL2L3, EAT, MCL1-ES, MCL1L, MCL1S, TM, bcl2-L-3 and mcl1/EAT); BCL2A1 (Genbank Accession Nos. NM_{—}001114735.1, NM_{—}004049.3, NP_{—}001108207.1 and NP_{—}004040.1; Aliases include: ACC-1, ACC-2, BCL2L5, BFL1, GRS and HBPA1); and BCL2L10 (Genbank Accession Nos. NM_{—}020396.2 and NP_{—}065129.1; Aliases include: BCL2-like 10). BCL-2 protein family polypeptides, e.g. those that are at least 50%, 55%, 60%, 65%, 70%, 75%, 80%, 85%, 90%, 91%, 92%, 95%, 97%, 99%, or are 100% identical to the sequence provided in the GenBank Accession Nos. above find use as conversion enhancing agents in the present invention, as do nucleic acids encoding these polypeptides or their antiapoptotic active domains and vectors comprising these nucleic acids.

In some instances, conversion enhancement agent is a member of the Inhibitor of Apoptosis (IAP) family. IAP family members may contain multiple baculovirus IAP repeat

(BIR) domains. The terms “IAP,” “IAP gene product,” “IAP polypeptide” and “IAP protein” are used interchangeably herein to refer to native sequence IAP protein family polypeptides, IAP protein family polypeptide variant, IAP protein family polypeptide fragments and chimeric IAP protein family polypeptides that can modulate apoptosis. Native sequence IAP protein family polypeptides include the proteins Survivin (Genbank Accession Nos. NM_{—}001012270.1, NM_{—}001012271.1, NM_{—}001168.2, NP_{—}001012270.1, NP_{—}001012271.1 and NP_{—}001159.2; Aliases include: API4, BIRC5, TIAP and EPR-1); XIAP (Genbank Accession Nos. NM_{—}001167.3, NM_{—}001204401.1, NP_{—}001158.2 and NP_{—}001191330.1; Aliases include: RP1-315G1.5, API3, BIRC4, IAP-3, ILP1, MIHA, XLP2, hIAP-3, hIAP3); BIRC2 (Genbank Accession Nos. NM_{—}001166.3 and NP_{—}001157.1 ; Aliases include: API1, HIAP2, Hiap-2, MIHB, RNF48, c-IAP1, cIAP1); BIRC3 (Genbank Accession Nos. NM_{—}001165.4, NM_{—}182962.2, NP_{—}001156.1 and NP_{—}892007.1; Aliases include: AIP1, API2, CIAP2, HAIP1, HIAP1, MALT2, MIHC, RNF49, c-IAP2); BIRC8 (Genbank Accession Nos. NM_{—}033341.4 and NP_{—}203127.3; Aliases include: ILP-2, ILP2, hILP2); BIRC7 (Genbank Accession Nos. NM_{—}022161.2, NP_{—}071444.1, NM_{—}139317.1, and NP_{—}647478.1; Aliases include: RP11-261N11.7, KIAP, LIVIN, ML-IAP, MLIAP, RNF50); NAIP (Genbank Accession Nos. NM_{—}004536.2, NM_{—}022892.1, NP_{—}004527.2 and NP_{—}075043.1; Aliases include: BIRC1, NLRB1, psiNAIP); and BIRC6 (Genbank Accession Nos. NM_{—}016252.3 and NP_{—}057336.3; Aliases include: APOLLON and BRUCE). IAP protein family polypeptides, e.g. those that are at least 50%, 55%, 60%, 65%, 70%, 75%, 80%, 85%, 90%, 91%, 92%, 95%, 97%, 99%, or are 100% identical to the sequence provided in the GenBank Accession Nos. above find use as conversion enhancing agents in the present invention, as do nucleic acids encoding these polypeptides or their antiapoptotic active domains and vectors comprising these nucleic acids.

Also of interest as conversion enhancing agents are compounds that exhibit cell death reducing activity, e.g., as defined above. As such, compounds of interest include, but are not limited to: IDN-6556 (3-{2-(2-tert-Butyl-phenylaminooxalyl)-amino]-propionylamino}-4-oxo-5-(2,3,5,6-tetrafluoro-phenoxy)-pentanoic Acid), IDN-1965 (N-[(1,3-dimethylindole-2-carbonyl)valinyl]-3-amino-4-oxo-5-fluoropentanoic acid), IDN-8066, IDN-7503, IDN-7436, M50054 (2,2′-Methylenebis(1,3-cyclohexanedione)), BAX Inhibiting Peptide V5, BTZO-1 (2-Pyridin-2-yl-4H-1,3-benzothiazin-4-one), Bongkrekic acid, MDL 28170 (Peptdie Z-Val-Phe-al), NS3694 (4-Chloro-2-[3-(3-trifluoromethyl-phenyl)-ureido]benzoic acid), NSCI (1-(4-Methoxybenzyl)-5-[2-(pyridin-3-yl-oxymethyl)pyrrolidine-1-sulfonyl]-1H-indole-2,3-dione), Necrostatin-1 (5-(1H-Indol-3-ylmethyl)-3-methyl-2-thioxo-4-Imidazolidinone 5-(Indol-3-ylmethyl)-3-methyl-2-thio-Hydantoin), 16F16 (2-(2-Chloroacetyl)-2,3,4,9-tetrahydro-1-methyl-1H-pyrido[3,4-b]indole-1-carboxylic acid methyl ester Methyl 2-(2-chloroacetyl)-1-methyl-2,3,4,9-tetrahydro-1H-pyrido[3,4-b]indole-1-carboxylate), Pifithrin-α (2-(2-Imino-4,5,6,7-tetrahydrobenzothiazol-3-yl)-1-p-tolylethanone hydrobromide), Pifithrin-p (2-Phenylethynesulfonamide) S-15176 difumarate salt (N-[(3,5-Di-tert-butyl-4-hydroxy-1-thiophenyl)]-3-propyl-N′-(2,3,4-trimethoxybenzyl)piperazine difumarate salt), Aurintricarboxylic Acid, and IN1407 ((+/−)-1-(3,6-Dibromocarbazol-9-yl)-3-piperazin-1-yl-propan-2-ol, bis TFA) (See Hoglen et al. (2004) J Pharmacol Exp Ther. May; 309(2):634-40; Hoglen et al. (2001) J Pharmacol Exp Ther. May; 297(2):811-8; Natori et al. (2003) Liver Transpl. March; 9(3):278-84;IDUN/Conatus Pharmaceuticals; and Sigma Aldrich).

In some embodiments, the one or more neurogenic factors, e.g. NeuroD2, and/or conversion enhancing agents, e.g., Bcl-XL, are provided as polypeptides. In other words, the subject cells are contacted with neurogenic factors and/or conversion enhancing agents that act in the appropriate subcellular domain. To promote transport of neurogenic factors and/or conversion enhancing agents across the cell membrane, the polypeptide sequences may be fused to a polypeptide permeant domain, e.g., peptide/protein transduction domains (PTDs). Any convenient permeant domain may be employed, where a number of permeant domains are known in the art and may be used, where such domains may be peptides, peptidomimetics, and non-peptide carriers. For example, a permeant peptide may be derived from the third alpha helix of Drosophila melanogaster transcription factor Antennapaedia, referred to as penetratin, which comprises the amino acid sequence RQIKIWFQNRRMKWKK (SEQ ID NO:135). As another example, the permeant peptide comprises the HIV-1 tat basic region amino acid sequence, which may include, for example, amino acids 49-57 of naturally-occurring tat protein. Other permeant domains include poly-arginine motifs, for example, the region of amino acids 34-56 of HIV-1 rev protein, nona-arginine, octa-arginine, and the like. (See, for example, Futaki et al. (2003) Curr Protein Pept Sci. 2003 April; 4(2): 87-96; and Wender et al. (2000) Proc. Natl. Acad. Sci. U.S.A 2000 Nov. 21; 97(24):13003-8; published U.S. Patent applications 20030220334; 20030083256; 20030032593; and 20030022831, herein specifically incorporated by reference for the teachings of translocation peptides and peptoids). The nona-arginine (R9) sequence is one of the more efficient PTDs that have been characterized (Wender et al. 2000; Uemura et al. 2002).

The polypeptides may be prepared by in vitro synthesis, using any convenient protocol such as conventional methods as known in the art. Various commercial synthetic apparatuses are available, for example, automated synthesizers by Applied Biosystems, Inc., Beckman, etc. By using synthesizers, naturally occurring amino acids may be substituted with unnatural amino acids. The particular sequence and the manner of preparation will be determined by convenience, economics, purity required, and the like. Other methods of preparing polypeptides in a cell-free system include, for example, those methods taught in U.S. Application Ser. No. 61/271,000, which is incorporated herein by reference.

The polypeptides may also be isolated and purified by using any convenient protocol, such as in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein. The polypeptides may be produced recombinantly not only directly, but also as a fusion polypeptide with a heterologous polypeptide, e.g. a polypeptide having a specific cleavage site at the N-terminus of the mature protein or polypeptide. Expression vectors usually contain a selection gene, also termed a selectable marker. This gene encodes a protein necessary for the survival or growth of transformed host cells grown in a selective culture medium.

Following purification, e.g., by commonly known methods in the art, the neurogenic factor and/or conversion enhancement agent polypeptides may be provided to the subject cells by standard protein transduction methods. In some cases, the protein transduction method includes contacting cells with a composition containing a carrier agent and at least one purified polypeptide. Examples of suitable carrier agents and methods for their use include, but are not limited to, commercially available reagents such as Chariot™ (Active Motif, Inc., Carlsbad, Calif.) described in U.S. Pat. No. 6,841,535; Bioport™ (Gene Therapy Systems, Inc., San Diego, Calif.), GenomeONE (Cosmo Bio Co., Ltd., Tokyo, Japan), and ProteoJuice™ (Novagen, Madison, Wis.), or nanoparticle protein transduction reagents as described in, e.g., U.S. patent application Ser. No. 10/138,593.

In other embodiments, the one or more neurogenic factors and/or conversion enhancing agents are provided in the cell by providing nucleic acids encoding polypeptide(s) of interest. These encoding nucleic acids may be provided in a cell using any convenient protocol, including those described above, e.g., direct introduction of nucleic acids into a cell, vector mediated introduction of the nucleic acids into the cell, etc.

In some instances, the target non-neuronal somatic cells include or have been modified to include expession cassettes encoding the various components or precursors thereof unter the control of an inducible expression system. Any convenient inducible expression system may be employed, where a variety of such systems are known in the art, e.g., the Tet-on inducible expression system. In these instances, the induction agent may be an inducer of the inducible expression system, e.g., tet, dox, etc.

When more than one component makes up the induction agent, e.g., where the induction agent includes two microRNAs and at least one neurogenic factor, the varioius components may be provided individually or as a single composition, that is, as a premixed composition, of components. The components may be added to the subject cells simultaneously or sequentially at different times. The components may be provided to non-neuronal somatic cells individually or as a single composition, that is, as a premixed composition, of components. The components may be provided at the same molar ratio or at different molar ratios. The components may be provided once or multiple times in the course of culturing the cells of the subject invention. For example, the components may be provided to the subject cells one or more times and the cells allowed to incubate with the components for some amount of time following each contacting event, e.g. 16-24 hours, after which time the media is replaced with fresh media and the cells are cultured further.

In addition to the induction agents (as well as optional neurogenic factors, conversion enhancing agents, etc., as desribed above, a given method may include use of other reagents. For example, a given method may include use of one or more agents that promote cell reprogramming. Examples of agents known in the art to promote cell reprogramming that may be employed include GSK-3 inhibitors (e.g. CHIR99021 and the like (see, e.g., Li, W. et al. (2009) Stem Cells, Epub Oct. 16 2009)); histone deacetylase (HDAC) inhibitors (e.g., those described in US20090191159, the disclosure of which is incorporated herein by reference); histone methyltransferase inhibitors (e.g. G9a histone methyltransferase inhibitors, e.g. BIX-01294, and the like (see, e.g. Shi, Yet al. (2008) Cell Stem Cells 3(5):568-574)); agonists of the dihydropyridine receptor (e.g. BayK8644, and the like (see, e.g., Shi, Y et al. (2008) Cell Stem Cell 3(5):568-574)); and inhibitors of TGFI3 signaling (e.g. RepSox and the like (see, e.g. Ichida, JK. et al. (2009) Cell Stem Cell 5(5):491-503)). Examples of agents known in the art to promote cell reprogramming also include agents that reduce the amount of methylated DNA in a cell, for example by suppressing DNA methylation activity in the cell or promoting DNA demethylation activity in a cell. Examples of agents that suppress DNA methylation activity include, e.g., agents that inhibit DNA methyltransferases (DNMTs), e.g. 5-aza-cytidine, 5-aza-2′-deoxycytidine, MG98, S-adenosyl-homocysteine (SAH) or an analogue thereof (e.g. periodate-oxidized adenosine or 3-deazaadenosine), DNA-based inhibitors such as those described in Bigey, P. et al (1999) J. Biol. Chem. 274:459-44606, antisense nucleotides such as those described in Ramchandani, S et al, (1997) Proc. Natl. Acad. Sci. USA 94: 684-689 and in Fournel, Met al, (1999) J. Biol. Chem. 274:24250-24256, or any other DNMT inhibitor known in the art. Examples of agents that promote DNA demethylation activity include, e.g., cytidine deaminases, e.g. AID/APOBEC agents (Bhutani, N et al. (2010) Nature 463(7284):1042-7; Rai, K. et al. (2008) Cell 135:1201-1212), agents that promote G:T mismatch-specific repair activity, e.g. Methyl binding domain proteins (e.g. Mbp4) and thymine-DNA glycosylase (TDG) protein (Rai, K. et al. (2008) Cell 135:1201-1212), agents that promote growth arrest and DNA-damage-inducible 45 (GADD45) activity protein (Rai, K. et al. (2008) Cell 135:1201-1212), and the like.

Other reagents of interest for optional inclusion in methods of invention include agents that promote the survival and differentiation of stem cells into neurons and/or mitotic neuronal progenitors or post-mitotic neuronal precursors into neurons. These types of agents include, for example, B27 (Invitrogen), glucose, transferrin, serum (e.g. fetal bovine serum, and the like), and the like. See, e.g. the Examples section presented below. Other reagents of interest for optional use in methods of the invention are agents that inhibit proliferation, e.g. AraC. Other reagents of interest for optional inclusion in methods of invention are agents that promote the differentiation of neuronal precursors into particular neuronal subtypes. For example, to promote differentiation into excitatory (glutamatergic) neurons, cells may also be contacted with Tlx polypeptides or nucleic acids encoding these polypeptides (e.g. Cheng, L. et al. (2004) Nat. Neurosci. 7(5):510-517). To promote differentiation into inhibitory (GABAergic) neurons, cells may also be contacted with Lbx1 polypeptides or nucleic acids encoding these polypeptides (e.g. Cheng, L. et al. (2005) Nature Neuroscience 8(11):1510-1515). To promote differentiation into dopaminergic (DA) neurons, cells may also be co-cultured with a PA6 mouse stromal cell line under serum-free conditions, see, e.g., Kawasaki et al., (2000) Neuron, 28(1):3140. To promote differentiation into cholinergic neurons, cells may also be contacted with Lhx8 polypeptides or nucleic acids encoding these polypeptides (Manabe, T. et al. (2007) Cell Death and Differentiation 14: 1080-1085). To promote differentiation of spinal cord motor neurons, cells may also be contacted with Mnx1 (Hb9) (Wichterle, H et al. (2002) Cell 110(3):385-397). To promote differentiation into corticospinal projection neurons, e.g. motor neurons, cells may also be contacted with Fezf2 or Ctip2 polypeptides or nucleic acids encoding those polypeptides (e.g. Molyneaux et al. (2005) Neuron 47(6):817-31; Chen et al. (2008) Proc Natl Acad Sci USA 105(32):11382-7). To promote differentiation of corticocortical projection neurons, e.g. callosal neurons, cells may be contacted with Satb2 polypeptides or nucleic acids encoding those polypeptides (e.g. Alcamo et al. (2008) Neuron 57(3):364-77; Britanova et al. (2008) Neuron 57(3):378-92). To promote differentiation of corticothalamic neurons, cells may be contacted with Sox5 polypeptides or nucleic acids encoding those polypeptides (e.g. Lai et al. (2008) Neuron 57(2):232-47). Other methods have also been described, see, e.g., Pomp et al., (2005), Stem Cells 23(7):923-30; U.S. Pat. No. 6,395,546, e.g., Lee et al., (2000), Nature Biotechnol., 18:675-679.

The various agents of the invention (and any optional reagents, as desired), e.g., as described above, may be provided in any convenient culture media, where culture media of interest include those that promote cell survival, e.g. DMEM, Iscoves, Neurobasal media, N3, etc. Media may be supplemented with agents that inhibit the growth of bacterial or yeast, e.g. penicillin/streptomycin, a fungicide, etc., with agents that promote health, e.g. glutamate, and other agents typically provided to culture media as are known in the art of tissue culture.

Non-induction agents of interest, e.g. conversion enhancing agents, agents that promote demethylation, agents that promote the survival and/or differentiation of neurons or subtypes of neurons, agents that inhibit proliferation, and the like, may be provided to the cells prior to providing the induction agent. Alternatively, they may be provided concurrently with providing the induction agent. Alternatively, they may be provided subsequently to providing the induction agent.

The induction agent is provided to non-neuronal somatic cells so as to reprogram, i.e.

convert, those cells into induced neuronal cells. Non-neuronal somatic cells include any somatic cell that would not give rise to a neuron in the absence of experimental manipulation. Examples of non-neuronal somatic cells include differentiating or differentiated cells from ectodermal (e.g.,keratinocytes), mesodermal (e.g.,fibroblast), endodermal (e.g., pancreatic cells), or neural crest lineages (e.g. melanocytes). The somatic cells may be, for example, pancreatic beta cells, glial cells (e.g. oligodendrocytes, astrocytes), hepatocytes, hepatic stem cells, cardiomyocytes, skeletal muscle cells, smooth muscle cells, hematopoietic cells, osteoclasts, osteoblasts, pericytes, vascular endothelial cells, schwann cells, dermal fibroblasts, and the like. They may be terminally differentiated cells, or they may be capable of giving rise to cells of a specific, non-neuronal lineage, e.g. cardiac stem cells, hepatic stem cells, and the like. The somatic cells are readily identifiable as non-neuronal by the absence of neuronal-specific markers that are well-known in the art, as described above. Of interest are cells that are vertebrate cells, e.g., mammalian cells, such as human cells, including adult human cells. In some instances, the non-neuronal somatic cells are glial cells (glia). The terms “glia” or “glial cells” refer to non-neuronal cells found in close contact with neurons, and encompass a number of different cells, including but not limited to the microglia, macroglia, neuroglia, astrocytes, astroglia, oligodendrocytes, ependymal cells, radial glia, Schwann cells, satellite cells, and enteric glial cells. Examples of markers that may be used to aid in the identification of glial cells include, but are not limited to Glial Fibrillary Acidic Protein (GFAP), 2′,3′-cyclic nucleotide 3′ phosphodiesterase (CNPase), myelin-associated glycoprotein (MAG), myelin basic protein (MBP), and S100 calcium binding protein B (s100B).

Embodiments of the invention may exhibit high conversion efficiency. By high conversion efficiency is meant that a substantial portion of the initial population of cells is converted to neuronal cells. By substantial portion is meant 25% by number or more, such as 40% by number or more, including 50% by number or more, such as 75% by number or more. In addition to enhancing the conversion of non-neuroal somatic cells into an induced neuronal cell using a method comprising microRNA, e.g., as described above, the high conversion efficiency achieved by using a conversion enhancement agent, e.g. BclXL, finds use in coverting non-neuroal somatic cells into an induced cell such as a neuronal cell, a neural stem cell, or a neural precursor cell employing methods that do not necessarily use microRNA. In some instances, such methods that do not use microRNA may instead employ proteins or nucleic acids that encode the same, such as transcription factors, including but not limited to: the conversion of fibroblasts into neural stem cells (by using at least one of Sox2, Klf4, c-Myc and Oct4, e.g., as reported in Their et al. (2012) Cell Stem Cell. 2012 Mar. 20); the conversion of fibroblasts into neural precursor cells (by using at least one of Brn2, Sox2, and FoxG1, e.g., as reported in Lujan et al. (2012) Proc Natl Acad Sci U S A. February 14; 109(7):2527-32); the conversion of hepatocytes into neurons (by using at least one of Ascl1, Brn2, and Myt11, e.g., as reported in Marro et al. (2011) Cell Stem Cell. October 4; 9(4):374-82); the conversion of fibroblasts into neurons (by using at least one of Ascl1, Brn2, and Myt1l, e.g., as reported in Vierbuchen et al. (2010) Nature. 2010 Feb. 25; 463(7284): 1035-41); the conversion of fibroblasts into spinal motor neurons (by using at least one of Ascl1, Brn2, Myt11, Lhx3, Hb9, Is11, Ngn2 and NeuroD1, e.g., as reported in Son et al. (2011) Cell Stem Cell. September 2; 9(3):205-18); the conversion of fibroblasts into dopaminergic neurons (by using at least one of Ascl1, Nurr1, and Lmx1 a, e.g., as reported in Caiazzo et al. (2011) Nature. 2011 Jul. 3; 476(7359):224-7; or at least one of Ascl1, Brn2, Myt1l, Lmx1 a, and FoxA2, e.g., as reported in Pfisterer et al. (2011) Proc Natl Acad Sci U S A. 2011 Jun. 21; 108(25):10343-8); the conversion of astroglia from the cerebral cortex into neurons (by using at least one of Neurog2, DIx2, and Mash1, e.g., as reported in Heinrich et al. (2010) PLoS Biol. 2010 May 18; 8(5):e1000373); and the conversion non-neuronal cells into iNs (by using at least one of an Ascl agent, a Ngn agent, a Brn agent, a NeuroD agent, a Myt1 agent, an Olig agent, and a Zic agent,e.g., as reported in PCT/U.S. Ser. No. 11/21731, herein specifically incorporated by reference).

In some embodiments, aspects of the invention include producing inhibitory neurons from non-neuronal cells, such as non-neuronal somatic cells, iPS cells, ES cells, etc. The term “inhibitory neuron” refers to a neuron that releases an inhibitory neurotransmitter to a nearby neuron such that the released inhibitory neurotransmitter exerts an inhibitory effect on the activity of said nearby neuron. By neurotransmitter is meant a molecule released by one neuron, thereby affecting the activity of a nearby neuron. The inhibitory neurotransmitter released by an inhibitory neuron produced according to embodiments of the inventionmay be gamma-aminobutyric acid (GABA), such that the inhibitory neuron that is produced may be a GABAergic neuron. In certain embodiments, the inhibitory neurotransmitter released by the inhibitory neurons produced by methods of the invention is glycine. Inhibitory neurons produced in accordance with the invention express, in some instances, vGAT, which is a protein specifically expressed by GABAergic inhibitory neurons, and the expression of vGAT by a neuron is commonly used in the art to characterize the neuron as an inhibitory neuron (Yoo et al., supra.).

### In Vitro Methods of Conversion and Uses for Cells Converted In Vitro

In some embodiments, the somatic cells are contacted in vitro with the induction agent. The somatic cells may be from any mammal, including humans, primates, domestic and farm animals, and zoo, laboratory or pet animals, such as dogs, cats, cattle, horses, sheep, pigs, goats, rabbits, rats, mice etc. They may be established cell lines or they may be primary cells, where “primary cells”, “primary cell lines”, and “primary cultures” are used interchangeably herein to refer to cells and cells cultures that have been derived from a subject and allowed to grow in vitro for a limited number of passages.

The subject cells may be isolated from fresh or frozen cells, which may be from a neonate, a juvenile or an adult, and from tissues including skin, muscle, bone marrow, peripheral blood, umbilical cord blood, spleen, liver, pancreas, lung, intestine, stomach, adipose, and other differentiated tissues. The tissue may be obtained by biopsy or aphoresis from a live donor, or obtained from a dead or dying donor within about 48 hours of death, or freshly frozen tissue, tissue frozen within about 12 hours of death and maintained at below about −20° C., usually at about liquid nitrogen temperature (-190° C.) indefinitely. For isolation of cells from tissue, an appropriate solution may be used for dispersion or suspension. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc. Cells contacted in vitro with the induction agent may be incubated in the presence of the agent for any convenient period, such as a period ranging from 30 minutes to 24 hours, e.g., 1 hours, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from 30 minutes to 24 hours, which may be repeated with a frequency of every day to every 4 days, e.g., every 1.5 days, every 2 days, every 3 days, or any other frequency from every day to every four days. The agent(s) may be provided to the subject cells one or more times, e.g. one time, twice, three times, or more than three times, and the cells allowed to incubate with the agent(s) for some amount of time following each contacting event e.g. 16-24 hours, after which time the media is replaced with fresh media and the cells are cultured further.

After contacting the non-neuronal somatic cells with the induction agent, the contacted cells may be cultured so as to promote the survival and differentiation of the induced neuronal cells of interest. Methods and reagents for culturing cells to promote the growth of neuronal cells or particular subtypes and for isolating neuronal cells of particular subtypes are well known in the art, any of which may be used in the present invention to grow and isolate the induced neuronal cells of interest. For example, the somatic cells (either pre- or post-contacting with the induction agent) may be plated on Matrigel or other substrate, e.g., as known in the art. The cells may be cultured in media such as N3, supplemented with factors. Alternatively, the contacted cells may be frozen at liquid nitrogen temperatures and stored for long periods of time, being capable of use on thawing. If frozen, the cells will usually be stored in a 10% DMSO, 50% FCS, 40% RPMI 1640 medium. Once thawed, the cells may be expanded by use of growth factors and/or stromal cells associated with neuronal survival and differentiation.

The effective amount of an induction agent that may used to contact the somatic cells is an amount that induces at least 0.01% of the cells of the culture to increase expression of one or more genes known in the art to become more highly expressed upon the acquisition of a neuronal fate, e.g. Tau, Tuj1, MAP2, NeuN, and the like. An effective amount is the amount that induces an increase in expression of these genes that is 1.5-fold or more, e.g. 1.5 fold, 2 fold, 3 fold, 4 fold, 6 fold, 10 fold greater (or more) than the level of expression observed in the absence of the induction agent. The level of gene expression can be readily determined by any of a number of well-known methods in the art, e.g. by measuring RNA levels, e.g. by RT-PCR, quantitative RT-PCR, Northern blot, etc., and by measuring protein levels, e.g. Western blot, ELISA, fluorescence activated cell sorting, etc.

It is noted here that the contacted somatic cells do not need to be cultured under methods known in the art to promote pluripotency in order to be converted into induced neuronal cells. By pluripotency, it is meant that the cells have the ability to differentiate into all types of cells in an organism. In other words, the methods of the present invention do not require that the somatic cells of the present invention be provided with reprogramming factors known in the art to reprogram somatic cells to become pluripotent stem cells, i.e. iPS cells, e.g. Oct3/4, SOX2, KLF4, MYC, Nanog, or Lin28, and be cultured under conditions known in the art to promote pluripotent stem cell induction, e.g., as hanging droplets, in order for the subject cells to be reprogrammed into induced neuronal (iN) cells. Following the methods of the invention, the contacted somatic cells will in some instances be converted into induced neuronal cells at an efficiency of reprogramming/efficiency of conversion that is 0.01% or more of the total number of somatic cells cultured initially, e.g., 0.01%, 0.05%, 0.1%, 0.5%, 1%, 2%, 3%, 4%, 5%, 6%, 7%, 8%, 9%, 10%, 12%, 14%, 16%, 20% or more. At times, depending on the age of the donor, the origin of the tissue, or the culture conditions, higher efficiencies may be achieved. This efficiency of reprogramming is an enhanced efficiency over that which may be observed in the absence of induction agent. In other words, somatic cells and cell cultures have an enhanced ability to give rise to the desired type of cell when contacted with one or more induction agents relative to cells that were not contacted with the induction agents. By enhanced, it is meant that the somatic cell cultures have the ability to give rise to the desired cell type that is 150% or greater than the ability of a somatic cell culture that was not contacted with the induction agent, e.g. 150%, 200%, 300%, 400%, 600%, 800%, 1000%, or 2000% of the ability of the uncontacted population. In other words, the culture of somatic cells produces about 1.5 fold, about 2-fold, about 3-fold, about 4-fold, about 6-fold, about 10-fold, about 20-fold, about 30-fold, about 50-fold, about 100-fold, about 200-fold or more the number of iN cells that are produced by a population of somatic cells that are not contacted with the induction agent. The efficiency of reprogramming may be determined by assaying the number of neuronal cells that develop in the cell culture, e.g. by assaying the number of cells that express genes that are expressed by neurons, e.g. Tau, Tuj1, MAP2, and/or NeuN, the number of cells that being to extend processes and make synaptic connections, the number of cells that may be depolarized and fire action potentials, e.g. single spikes or a train of action potentials, etc.

Induced neuronal (iN) cells produced by the above in vitro methods may be used in cell replacement therapy to treat diseases. Specifically, iN cells may be transferred to subjects suffering from a wide range of diseases or disorders with a neuronal component, i.e. with neuronal symptoms, for example to reconstitute or supplement differentiating or differentiated neurons in a recipient.

Therapy may be directed at treating the cause of the disease; or alternatively, therapy may be to treat the effects of the disease or condition. For example, therapy may be directed at replacing neurons whose death caused the disease, e.g. motor neurons in Amyotrophic lateral sclerosis (ALS), or therapy may be directed at replacing neurons that died as a result of the disease, e.g. photoreceptors in age related macular degeneration (AMD).

The iN cells may be transferred to, or close to, an injured site in a subject; or the cells can be introduced to the subject in a manner allowing the cells to migrate, or home, to the injured site. The transferred cells may advantageously replace the damaged or injured cells and allow improvement in the overall condition of the subject. In some instances, the transferred cells may stimulate tissue regeneration or repair.

In some cases, the iN cells or a sub-population of iN cells may be purified or isolated from the rest of the cell culture prior to transferring to the subject. In other words, one or more steps may be executed to enrich for the iN cells or a subpopulation of iN cells, i.e. to provide an enriched population of iN cells or subpopulation of iN cells. In some cases, one or more antibodies specific for a marker of cells of the neuronal lineage or a marker of a sub-population of cells of the neuronal lineage are incubated with the cell population and those bound cells are isolated. In other cases, the iN cells or a sub-population of the iN cells express a marker that is a reporter gene, e.g. EGFP, dsRED, lacz, and the like, that is under the control of a neuron-specific promoter or neuron-subtype specific promoter, e.g. Tau, GABA, NMDA, and the like, which is then used to purify or isolate the iN cells or a subpopulation thereof.

By a marker it is meant that, in cultures comprising somatic cells that have been reprogrammed to become iN cells, the marker is expressed only by the cells of the culture that will develop, are developing, and/or have developed into neurons. It will be understood by those of skill in the art that the stated expression levels reflect detectable amounts of the marker protein on or in the cell. A cell that is negative for staining (the level of binding of a marker-specific reagent is not detectably different from an isotype matched control) may still express minor amounts of the marker. And while it is commonplace in the art to refer to cells as “positive” or “negative” for a particular marker, actual expression levels are a quantitative trait. The number of molecules on the cell surface can vary by several logs, yet still be characterized as “positive”.

Cells of interest, e.g., cells expressing the marker of choice, may be enriched for, that is, separated from the rest of the cell population, by any convenient protocol. For example, flow cytometry, e.g., fluorescence activated cell sorting (FACS), may be used to separate the cell population based on the intrinsic fluorescence of the marker, or the binding of the marker to a specific fluorescent reagent, e.g. a fluorophor-conjugated antibody, as well as other parameters such as cell size and light scatter. In other words, selection of the cells may be effected by flow cytometry. Although the absolute level of staining may differ with a particular fluorochrome and reagent preparation, the data can be normalized to a control. To normalize the distribution to a control, each cell is recorded as a data point having a particular intensity of staining. These data points may be displayed according to a log scale, where the unit of measure is arbitrary staining intensity. In one example, the brightest stained cells in a sample can be as much as 4 logs more intense than unstained cells. When displayed in this manner, it is clear that the cells falling in the highest log of staining intensity are bright, while those in the lowest intensity are negative. The “low” positively stained cells have a level of staining above the brightness of an isotype matched control, but are not as intense as the most brightly staining cells normally found in the population. An alternative control may utilize a substrate having a defined density of marker on its surface, for example a fabricated bead or cell line, which provides the positive control for intensity. Other methods of separation, e.g., methods by which selection of cells may be effected, based upon markers include, for example, magnetic activated cell sorting (MACS), immunopanning, and laser capture microdissection.

One example of a protein of interest that may be used as a marker in such embodiments is PSA-NCAM. PSA-NCAM is an NCAM polypeptide (GenBank Accession Nos. NM_{—}000615.5 (isoform 1), NM_{—}181351.3 (isoform 2) and NM_{—}001076682.2 (isoform 3)), that is post-translationally modified by the addition of poly-sialic acid. A number of antibodies that are specific for PSA-NCAM are known in the art, including, e.g., anti-PSA-NCAM Clone 2-2B antibody (Millipore). Another example of a marker that may be used is a fluorescent protein, e.g. GFP, RFP, dsRED, etc., operably linked to a neuron-specific promoter, e.g. Tau, PSA-NCAM, etc. In such embodiments, the marker and promoter are provided to the cell as an expression cassette on a vector, e.g. encoded on a DNA plasmid, encoded in a virus, and the like. The expression cassette may optionally contain other elements, e.g. enhancer sequences, other proteins for expression in the cell, and the like. In some embodiments, the expression cassette is provided to the cell prior to contacting the cell with the induction agent, i.e. while the cell is still a somatic cell. In some embodiments, the expression cassette is provided to the cell at the same time as the cell is contacted with the induction agent. In some embodiments, the expression cassette is provided to the cell after the cell is contacted with the induction agent.

Enrichment of the iN population or a subpopulation of iNs may be performed at a suitable time following contact of the cells with the induction agent, such as 3 days or more, e.g. 4 days or more, 5 days or more, 6 days or more, 7 days or more, 10 days or more, 14 days or more, or 21 days or more after contacting the somatic cells with the induction agent.

Populations that are enriched by selecting for the expression of one or more markers will usually have at 80% or more cells of the selected phenotype, such as 90% or more cells and including 95% or more of the cells of the selected phenotype.

In some cases, genes may be introduced into the somatic cells or the cells derived therefrom, i.e. iNs, prior to transferring to a subject for a variety of purposes, e.g. to replace genes having a loss of function mutation, provide marker genes, etc. Alternatively, vectors are introduced that express antisense mRNA or ribozymes, thereby blocking expression of an undesired gene. Other methods of gene therapy are the introduction of drug resistance genes to enable normal progenitor cells to have an advantage and be subject to selective pressure, for example the multiple drug resistance gene (MDR), or anti-apoptosis genes, such as bcl-2. Various techniques known in the art may be used to introduce nucleic acids into the target cells, e.g. electroporation, calcium precipitated DNA, fusion, transfection, lipofection, infection and the like, as discussed above. The particular manner in which the DNA is introduced is not critical to the practice of the invention.

To prove that one has genetically modified the somatic cells or the cells derived therefrom, i.e., iNs, various techniques may be employed. The genome of the cells may be restricted and used with or without amplification. The polymerase chain reaction; gel electrophoresis; restriction analysis; Southern, Northern, and Western blots; sequencing; or the like, may all be employed. The cells may be grown under various conditions to ensure that the cells are capable of maturation to all of the neuronal lineages while maintaining the ability to express the introduced DNA. Various tests in vitro and in vivo may be employed to ensure that the neuronal phenotype of the derived cells has been maintained.

Subjects in need of neuron replacement therapy, e.g., a subject suffering from a neurological condition associated with the loss of neurons or with aberrantly functioning neurons, could especially benefit from therapies that utilize cells derived by the methods of the invention. Examples of such diseases, disorders and conditions include neurodegenerative diseases (e.g. Parkinson's Disease, Alzheimer's Disease, Huntington's Disease, Amyotrophic Lateral Sclerosis (ALS), Spielmeyer-Vogt-Sjogren-Batten disease (Batten Disease), Frontotemporal Dementia with Parkinsonism, Progressive Supranuclear Palsy, Pick Disease, prion diseases (e.g. Creutzfeldt-Jakob disease), Amyloidosis, glaucoma, diabetic retinopathy, age related macular degeneration (AMD), and the like); neuropsychiatric disorders (e.g. anxiety disorders (e.g. obsessive compulsive disorder), mood disorders (e.g. depression), childhood disorders (e.g. attention deficit disorder, autistic disorders), cognitive disorders (e.g. delirium, dementia), schizophrenia, substance related disorders (e.g. addiction), eating disorders, and the like); channelopathies (e.g. epilepsy, migraine, and the like); lysosomal storage disorders (e.g. Tay-Sachs disease, Gaucher disease, Fabry disease, Pompe disease, Niemann-Pick disease, Mucopolysaccharidosis (MPS) & related diseases, and the like); autoimmune diseases of the CNS (e.g. Multiple Sclerosis, encephalomyelitis, paraneoplastic syndromes (e.g. cerebellar degeneration), autoimmune inner ear disease, opsoclonus myoclonus syndrome, and the like); cerebral infarction, stroke, and spinal cord injury.

In some approaches, the reprogrammed somatic cells, i.e. iNs, may be transplanted directly to an injured site to treat a neurological condition, see, e.g., Morizane et al., (2008), Cell Tissue Res., 331(1):323-326; Coutts and Keirstead (2008), Exp. Neurol., 209(2):368-377; Goswami and Rao (2007), Drugs, 10(10):713-719. For example, for the treatment of Parkinson's disease, neurons may be transplanted directly into the striate body of a subject with Parkinson's disease. As another example, for treatment of ALS, corticospinal motor neurons may be transplanted directly into the motor cortex of the subject with ALS. In other approaches, the cells derived by the methods of the invention are engineered to respond to cues that can target their migration into lesions for brain and spinal cord repair; see, e.g., Chen et al. (2007) Stem Cell Rev. 3(4):280-288.

The iNs may be administered in any physiologically acceptable medium. They may be provided prior to differentiation, i.e. they may be provided in an undifferentiated state and allowed to differentiate in vivo, or they may be allowed to differentiate for a period of time ex vivo and provided following differentiation. They may be provided alone or with a suitable substrate or matrix, e.g. to support their growth and/or organization in the tissue to which they are being transplanted. In some instances, 1×10⁵ or more cells will be administered, such as 1×10⁶ or more cells. The cells may be introduced to the subject via any convenient protocol, including but not limited to: parenteral, intravenous, intracranial, intraspinal, intraocular, or into spinal fluid. The cells may be introduced by injection, catheter, or the like. Examples of methods for local delivery, that is, delivery to the site of injury, include, e.g. through an Ommaya reservoir, e.g. for intrathecal delivery (see e.g. U.S. Pat. Nos. 5,222,982 and 5385582, incorporated herein by reference); by bolus injection, e.g. by a syringe, e.g. intravitreally or intracranially; by continuous infusion, e.g. by cannulation, e.g. with convection (see e.g. US Application No. 20070254842, incorporated here by reference); or by implanting a device upon which the cells have been reversably affixed (see e.g. US Application Nos. 20080081064 and 20090196903, incorporated herein by reference).

The number of administrations of treatment to a subject may vary. Introducing the iNs into the subject may be a one-time event; but in certain situations, such treatment may elicit improvement for a limited period of time and require an on-going series of repeated treatments. In other situations, multiple administrations of the iNs may be required before an effect is observed. The exact protocols depend upon the disease or condition, the stage of the disease and parameters of the individual subject being treated.

Additionally or alternatively, iNs produced by the above in vitro methods may be used as a basic research or drug discovery tool, for example to evaluate the phenotype of a genetic disease, e.g. to better understand the etiology of the disease, to identify target proteins for therapeutic treatment, to identify candidate agents with disease-modifying activity, i.e. an activity in modulating the survival or function of neurons in a subject suffering from a neurological disease or disorder, e.g. to identify an agent that will be efficacious in treating the subject. For example, a candidate agent may be added to a cell culture comprising iNs derived from the subject's somatic cells, and the effect of the candidate agent assessed by monitoring output parameters such as iN survival, the ability of the iNs to become depolarized, the extent to which the iNs form synapses, and the like, by methods described herein and in the art.

Parameters are quantifiable components of cells, particularly components that can be accurately measured, desirably in a high throughput system. A parameter can be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values.

Candidate agents of interest for screening include known and unknown compounds that encompass numerous chemical classes, primarily organic molecules, which may include organometallic molecules, inorganic molecules, genetic sequences, etc. An important aspect of the invention is to evaluate candidate drugs, including toxicity testing; and the like. Candidate agents include organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof. Included are pharmacologically active drugs, genetically active molecules, etc. Compounds of interest include chemotherapeutic agents, hormones or hormone antagonists, etc. Exemplary of pharmaceutical agents suitable for this invention are those described in, “The Pharmacological Basis of Therapeutics,” Goodman and Gilman, McGraw-Hill, New York, N.Y., (1996), Ninth edition.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Candidate agents are screened for biological activity by adding the agent to one or a plurality of cell samples, usually in conjunction with cells lacking the agent. The change in parameters in response to the agent is measured, and the result evaluated by comparison to reference cultures, e.g. in the presence and absence of the agent, obtained with other agents, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

### In Vivo Methods of Conversion and Uses for Cells Converted In Vivo

In some embodiments, a somatic cell is contacted in vivo with the induction agent, e.g. in a subject in need of neuron replacement therapy. Cells in vivo may be contacted with an induction agent, e.g., in the form of a pharmaceutical composition, using any convenient protocol. The induction agent pharmaceutical composition can be incorporated into a variety of formulations. More particularly, the induction agent pharmaceutical composition can be formulated into pharmaceutical compositions by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the induction agent pharmaceutical composition can be achieved in various ways, including oral, buccal, rectal, parenteral, intraperitoneal, intradermal, transdermal, intracheal, etc., administration. The induction agent pharmaceutical composition may be systemic after administration or may be localized by the use of regional administration, intramural administration, or use of an implant that acts to retain the active dose at the site of implantation. The induction agent pharmaceutical composition may be formulated for immediate activity or they may be formulated for sustained release.

For some central nervous system conditions, it may be necessary to formulate the induction agent pharmaceutical composition to cross the blood brain barrier (BBB). One strategy for drug delivery through the blood brain barrier (BBB) entails disruption of the BBB, either by osmotic means such as mannitol or leukotrienes, or biochemically by the use of vasoactive substances such as bradykinin. A BBB disrupting agent can be co-administered with therapeutic compositions of the invention when the compositions are administered by intravascular injection. Other strategies to go through the BBB may entail the use of endogenous transport systems, including caveoil-1 mediated transcytosis, carrier-mediated transporters such as glucose and amino acid carriers, receptor-mediated transcytosis for insulin or transferrin, and active efflux transporters such as p-glycoprotein. Active transport moieties may also be conjugated to therapeutic compounds for use in the invention to facilitate transport across the endothelial wall of the blood vessel. Alternatively, drug delivery of the induction agent pharmaceutical composition behind the BBB may be by local delivery, for example by intrathecal delivery, e.g. through an Ommaya reservoir (see e.g. U.S. Pat. Nos. 5,222,982 and 5385582, incorporated herein by reference); by bolus injection, e.g. by a syringe, e.g. intravitreally or intracranially; by continuous infusion, e.g. by cannulation, e.g. with convection (see e.g. US Application No. 20070254842, incorporated here by reference); or by implanting a device upon which the induction agent pharmaceutical composition has been reversably affixed (see e.g. US Application Nos. 20080081064 and 20090196903, incorporated herein by reference).

The calculation of the effective amount or effective dose of the induction agent pharmaceutical composition to be administered is within the skill of one of ordinary skill in the art, and will be routine to those persons skilled in the art. The final amount to be administered will be dependent upon the route of administration and upon the nature of the disorder or condition that is to be treated.

For inclusion in a medicament, the induction agent pharmaceutical composition may be obtained from a suitable commercial source. As a general proposition, the total pharmaceutically effective amount of the compound administered parenterally per dose will be in a range that can be measured by a dose response curve.

The induction agent pharmaceutical composition to be used for therapeutic administration must be sterile. Sterility is readily accomplished by filtration through sterile filtration membranes (e.g., 0.2 pm membranes). Therapeutic compositions generally are placed into a container having a sterile access port, for example, an intravenous solution bag or vial having a stopper pierceable by a hypodermic injection needle. The induction agent pharmaceutical composition ordinarily will be stored in unit or multi-dose containers, for example, sealed ampules or vials, as an aqueous solution or as a lyophilized formulation for reconstitution. As an example of a lyophilized formulation, 10-mL vials are filled with 5 ml of sterile-filtered 1% (w/v) aqueous solution of compound, and the resulting mixture is lyophilized. The pharmaceutical composition comprising the lyophilized induction agent is prepared by reconstituting the lyophilized compound, for example, by using bacteriostatic Water-for-Injection.

An induction agent system for pharmaceutical use, e.g., an induction agent pharmaceutical composition, can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the induction agent pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The induction agent of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The induction agent composition can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is therapeutic index and it can be expressed as the ratio LD₅₀/ED₅₀. Compounds that exhibit large therapeutic indices are preferred. The data obtained from cell culture and/or animal studies can be used in formulating a range of dosages for humans. The dosage of the active ingredient typically lines within a range of circulating concentrations that include the ED₅₀ with low toxicity. The dosage can vary within this range depending upon the dosage form employed and the route of administration utilized.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The effective amount of a therapeutic composition to be given to a particular patient will depend on a variety of factors, several of which will differ from patient to patient. A competent clinician will be able to determine an effective amount of a therapeutic agent to administer to a patient to halt or reverse the progression the disease condition as required. Utilizing LD₅₀ animal data, and other information available for the agent, a clinician can determine the maximum safe dose for an individual, depending on the route of administration. For instance, an intravenously administered dose may be more than an intrathecally administered dose, given the greater body of fluid into which therapeutic composition is being administered. Similarly, compositions which are rapidly cleared from the body may be administered at higher doses, or in repeated doses, in order to maintain a therapeutic concentration. Utilizing ordinary skill, the competent clinician will be able to optimize the dosage of a particular therapeutic in the course of routine clinical trials.

Mammalian species that may be treated with the present methods include canines and felines; equines; bovines; ovines; etc. and primates, particularly humans. Animal models, particularly small mammals, e.g. murine, lagomorpha, etc. may be used for experimental investigations.

More particularly, the present invention finds use in the treatment of subjects, such as human patients, in need of neuron replacement therapy. Examples of such subjects would be subjects suffering from conditions associated with the loss of neurons or with aberrantly functioning neurons. Patients having diseases and disorders characterized by such conditions will benefit greatly by a treatment protocol of the pending claimed invention. Examples of such diseases, disorders and conditions include e.g., neurodegenerative diseases, neuropsychiatric disorders, channelopathies, lysosomal storage disorders, autoimmune diseases of the CNS, cerebral infarction, stroke, and spinal cord injury, as described previously.

In some instances, the somatic cell(s) contacted in vivo with at least a neuronal cell induction agentis a glial cell or a population of glial cells. As such, in vivo methods e.g., as described above, may be employed to convert glial cells to neuronal cells by contacting said glial cells in vivo with at least a neuronal cell induction agent. In some instances, at least one conversion enhancement agent may also be used, i.e. the glial cells may be contacted in vivo with at least one conversion enhancement agent. Glial cells, defined above, are abundant throughout the body and are found in close contact with neurons and provide a convenient source of non-neuronal somatic cells for conversion into neurons in vivo. Embodiments of such methods find use in a vareity of different applications, including but not limited to, the treatment and/or prevention of the aforementioned diseases, disorders and conditions. For example, methods of the invention may be employed to produce Dopaminergic neurons from local glia in the treatment of diseases such as Parkinson's disease.

An effective amount of an induction agent pharmaceutical composition is the amount that will result in an increase the number of neurons at the site of injury, and/or will result in measurable reduction in the rate of disease progression in vivo. For example, an effective amount of an induction agent pharmaceutical composition will inhibit the progression of symptoms e.g. loss of muscle control, loss of cognition, hearing loss, vision loss, etc. by at least about 5%, at least about 10%, at least about 20%, preferably from about 20% to about 50%, and even more preferably, by greater than 50% (e.g., from about 50% to about 100%) as compared to the appropriate control, the control typically being a subject not treated with the induction agent pharmaceutical composition. An agent is effective in vivo if administration of the agent at about 1 μg/kg to about 100 mg/kg body weight results in inhibition of symptoms within about 1 month to 3 months from the first administration of the pharmaceutical composition. In a specific aspect, body function may be improved relative to the amount of function observed at the start of therapy.

The methods of the present invention also find use in combined therapies, e.g. in with therapies that are already known in the art to provide relief from symptoms associated with the aforementioned diseases, disorders and conditions. The combined use of an induction agent pharmaceutical composition of the present invention and these other agents may have the advantages that the required dosages for the individual drugs is lower, and the effect of the different drugs complementary.

### Screening Methods

The methods described herein also provide a useful system for screening candidate agents for activity in modulating somatic cell conversion into somatic cells of a different cell lineage, e.g. neurons. In screening assays for biologically active agents, cells, usually cultures of cells, are contacted with a candidate agent of interest in the presence of the somatic cell reprogramming system or an incomplete somatic cell reprogramming system, and the effect of the candidate agent is assessed by monitoring output parameters such as the level of expression of genes specific for the desired cell type, e.g., neuron, or the ability of the cells that are induced to function like the desired cell type, e.g. to propagate an action potential (for neurons); etc.

For example, agents can be screened for an activity in promoting reprogramming of somatic cells to a neuronal cell fate. For such a screen, a candidate agent may be added to a cell culture comprising somatic cells and an induction agent or an incomplete induction agent, where an observed increase in the level of RNA or protein of a neuronal gene, e.g. a 1.5-fold, a 2-fold, a 3-fold or more increase in the amount of RNA or protein from a neuronal-specific gene, e.g., Tau, Beta-III-Tubulin (encoding the protein Tuj1), MAP2, and the like, over that observed in the culture absent the candidate agent would be an indication that the candidate agent was an agent that promotes reprogramming to a neuronal fate. Reciprocally, an observed decrease in the level of RNA or protein of a neuronal gene, e.g. a 1.5-fold, a 2-fold, a 3-fold or more decrease in the amount of RNA or protein from a neuronal-specific gene, e.g., Tau, Tuji, MAP2, as compared to that observed in the culture absent the candidate agent would be an indication that the candidate agent was an agent that suppresses reprogramming to a neuronal fate. Incomplete induction agents, e.g. an induction agent lacking one or more components, or comprising sub-optimal levels of one or more components, and the like, may be used in place of a complete induction agent to increase the sensitivity of the screen.

As another example, agents can be screened for an activity in promoting the development of a neuron derived from a reprogrammed somatic cell, e.g. the development of synapses by a neuron derived from a reprogrammed somatic cell. In such a case, a candidate agent may be added to a cell culture comprising newly-induced neurons, e.g. neurons that were induced from somatic cells by contacting the somatic cells with and induction agent 3 days, 4 days, 5 days, 6 days, 7 days or 10 days or more prior to contacting with the candidate agent. In some embodiments, the induced neurons are purified/isolated from the induction agent-contacted culture and replated prior to contacting with the candidate agent, e.g. by methods described above for enriching for iN cells. In some embodiments, the induced neurons are contacted with the candidate agent in the context of the induction agent, e.g. 2 days, 3 days, 5 days, 7 days or 10 days or more after the initial contact with the induction agent. For example, in a screen of candidate agents that modulate synapse development, an observed increase in the spontaneous and rhythmic network activity at a holding potential of −70 mV, in the number of excitatory (EPSC) and inhibitory (IPSO) postsynaptic currents evoked, or in the number of synapsin-positive puncta surrounding MAP-2 positive dendrites as observed by immunohistochemistry, e.g. a 1.5-fold, a 2-fold, a 3-fold or more increase in these parameters, over that observed in the culture absent the candidate agent would be an indication that the candidate agent was an agent that promotes synapse formation. Reciprocally, an observed decrease in the spontaneous and rhythmic network activity at a holding potential of −70 mV, in the number of excitatory (EPSC) and inhibitory (IPSO) postsynaptic currents evoked, or in the number of synapsin-positive puncta surrounding MAP-2 positive dendrites as observed by immunohistochemistry, e.g. a 1.5-fold, a 2-fold, a 3-fold or more decrease in these parameters, as compared to that observed in the culture absent the candidate agent would be an indication that the candidate agent was an agent that suppresses synapse formation.

As discussed above with regard to uses for iNs produced by in vitro methods in screening candidate agents for those with an activity in modulating the survival or activity of neurons in a subject suffering from a neurological disease or disorder, candidate agents of interest for screening include known and unknown compounds that encompass numerous chemical classes, primarily organic molecules, which may include organometallic molecules, inorganic molecules, genetic sequences, etc. An important aspect of the invention is to evaluate candidate drugs, including toxicity testing; and the like.

Also as discussed above, compounds, including candidate agents, may be obtained from a wide variety of sources including libraries of synthetic or natural compounds. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Also as discussed above, candidate agents are screened for biological activity by adding the agent to one or a plurality of cell samples, usually in conjunction with cells lacking the agent. The change in parameters in response to the agent is measured, and the result evaluated by comparison to reference cultures, e.g. in the presence and absence of the agent, obtained with other agents, etc. As discussed above, the agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

Various methods can be utilized for quantifying the chosen parameters. For example, a convention method of measuring the presence or amount of a selected marker is to label a molecule with a detectable moiety, which may be fluorescent, luminescent, radioactive, enzymatically active, etc., particularly a molecule specific for binding to the parameter with high affinity. Fluorescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to autofluoresce, e.g. by expressing them as green fluorescent protein chimeras inside cells (for a review see Jones et al. (1999) Trends Biotechnol. 17(12):477-81).

### Kits

Kits may be provided, where the kit will comprise one or more components of the induction agent to promote the direct conversion of somatic cells neuronal cells. Any of the components described above may be provided in the kits, e.g., the specific microRNAs or vectors comprising expression cassettes encoding the same or precursors thereof, the specific neurogenic factors described above or expression cassettes encoding the same, the specific conversion enhancing agents described above or expression cassettes encoding the same, etc. Kits may further include somatic cells or reagents suitable for isolating and culturing primary somatic cells in preparation for conversion; reagents suitable for culturing neurons; and reagents useful for determining the expression of neuron-specific genes in the contacted cells. Kits may also include tubes, buffers, etc., and instructions for use. The various reagent components of the kits may be present in separate containers, or some or all of them may be pre-combined into a reagent mixture in a single container, as desired.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another form of these instructions is a computer readable medium, e.g., diskette, compact disk (CD), etc., on which the information has been recorded. Yet another form of these instructions that may be present is a website address which may be used via the internet to access the information at a removed site.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### I. MATERIALS AND METHODS

A synthetic cluster of miR-9/9* and miR-124 validated previously to overexpress miR-9* and miR-124 (Yoo, A. S., Staahl, B. T., Chen, L., & Crabtree, G. R., MicroRNA-mediated switching of chromatin-remodelling complexes in neural development. Nature 460 (7255), 642-646 (2009)) was inserted downstream of turboRFP in the pLemiR lentiviral construct carrying a puromycin selection cassette (Openbiosystems) driven by either CMV promoter or doxycycline-reponsive promoter. A non-silencing sequence, which produces non-specific microRNA, was used as a control (Openbiosystems). Human NeuroD2 cDNA (as well as other neural transcription factors) was cloned downstream of the EF1alpha promoter in a separate lentiviral construct with blasticidin selection. BclXL cDNA was cloned downstream of a Dox-inducible promoter. In a typical experiment, infected human fibroblasts were maintained in fibroblast media for 2 days before selection with appropriate antibiotics in RHA-B media (StemCell Inc.) with 2% FBS (Hyclone), valproic acid (VPA, 1 mM), retinoic acid (RA, 2 mM), bFGF (20 ng/ml) and EGF (20 ng/ml). 7 days after infection, the media was changed to Neuronal Media (ScienCell) supplemented with VPA (1 mM), RA (2 mM), bFGF (20 ng/ml) and dbcAMP (100 μM) until the end of the experiments. Human BDNF and GDNF (10 ng/ml, Peprotech) were added to the media after two weeks. The media was changed every 4 days.

### A. Plasmid Construction and Viral Preparation

We have previously constructed a synthetic cluster that expresses the precursors of both miR-9/9* and miR-124 and validated its ability to generate mature miRNAs of both (Yoo et al., supra.) Here we cloned this cluster downstream of a turbo red fluorescent protein (tRFP) marker into the lentiviral pLemiR (Openbiosystems), driven by either CMV promoter or doxycycline-reponsive promoter. This construct also carries a puromycin resistance cassette to allow for both visual tracking as well as antibiotic selection of the infected cells. A non-silencing sequence in pLemiR (which produces non-specific microRNA) was used as a control (Openbiosystems). To allow for selection of dually-infected cells, complementary DNA of human NeuroD2 and other neural factors (Openbiosystems) was cloned downstream of the EF1 a promoter in a separate lentiviral construct with blasticidin selection. BclXL cDNA was cloned downstream of a Dox-inducible promoter. Infectious lentiviruses were collected 36-60 hours after transfection of Lenti-X 293T cells (Clontech) with appropriate amounts of lentiviral vectors, psPAX2 and pMD2.G (Addgene) using Fugene HD (Roche).

### B. Cell Culture

All fibroblast cultures (human neonatal foreskin fibroblasts (ATCC, PCS-201-010), SS neonatal foreskin fibroblasts (derived in our lab) and adult dermal fibroblast (ScienCell)) were maintained in fibroblast media (Dulbecco's Modified Eagle Medium; Invitrogen) containing 10% fetal bovine serum (FBS, Omega Scientific), β-mercaptoethanol (Sigma-Aldrich), non-essential amino acids, sodium pyruvate, glutamax, and penicillin/streptomycin (all from Invitrogen). The day before lentiviral infection, human fibroblasts were seeded onto poly-D-Lysine (Sigma-Aldrich)/Laminin (Roche)/Fibronectin (Sigma-Aldrich) coated 24-well tissue culture dishes (MidSci). Next day, cells were infected with filtered viral supernatants in the presence of polybrene (8 μg/ml) over night. Fresh media were then replaced for 2 days after the infection before switching to RHB-A media (StemCells Inc) with appropriate antibiotics to select for infected cells, in the presence of 2% FBS (Hyclone) VPA (1 mM), RA (5 μM), bFGF (20 ng/ml,) and EGF (20 ng/ml). 7 days post infection, the media were changed to Neuronal Media (ScienCell) supplemented with VPA (1 mM), RA (5 μM), bFGF (20 ng/ml) until the end of the experiments, with media changes every 4 days. Human BDNF and GDNF (10 ng/ml, Peprotech) were added to the media after two weeks. To facilitate immunostaining and electrophysiological studies in some experiments cells were trypnized (0.05% Trypsin, Invitrogen) at about 7-day post infection and re-plated onto poly-D-lysine/laminin/fibronectin coated glass coverslips.

For experiments involving the expression of BclXL, the conversion of glial cells to neurons, and conversion of fibroblasts to inhibitory neurons, primary human fibroblasts were maintained in fibroblast media (Dulbecco's Modified Eagle Medium; Invitrogen) containing 10% fetal bovine serum (FBS, Omega Scientific), β-mercaptoethanol (Sigma-Aldrich), non-essential amino acids, sodium pyruvate, glutamate, and penicillin/streptomycin (all from Invitrogen). Human glial cells, obtained fom different commercial sources were maintained in astrocyte media lacking FCS. 3-4 days post infection media were changed to Neuronal Media (ScienCell) with VPA (1 mM). dbcAMP (500 mM) was added 15 days later to enhance cell survival. Human BDNF and NT3 (10 ng ml21; Peprotech) were added to the media after 3-4 weeks. Media were changed every 4 days.

### C. Immunofluorescence

The following antibodies were used for the immunofluorescence studies: mouse anti-MAP2 (Sigma-Aldrich, 1:750), chicken anti-MAP2 (Abcam, 1: 30,000), mouse anti-b-III tubulin (Covance, 1: 30,000), rabbit anti-VGLUT1 (Synaptic Systems, 1: 2000), rabbit anti-TBR1 (Abcam, 1:500), rabbit anti-Scn1a (Abcam, 1:1000), rabbit anti-NMDAR1 (1: 2000), rabbit anti-Neurofilament 200 (Sigma-Aldrich, 1:2000), rabbit anti-Synapsin1 (Cell Signaling, 1:200) and anti-GABA. Antibodies against BAF subunits were generated in our lab and used as the following concentrations: BAF45b (1: 250), BAF45c (1: 1000) and BAF53b (1: 500). The secondary antibodies were goat anti-rabbit or mouse IgG conjugated with Alexa-488 or -647 (Invitrogen). For Scn1 a and BAF53b staining, biotinylated secondary antibodies were detected using TSA amplification kit (Invitrogen). EdU incorporation assay was performed according to the manufacture's protocols (Invitrogen). Images were captured using Leica DM5000B microscope with Leica Application Suite (LAS) Advanced Fluorescence 1.8.0 and Leica DMI4000B microscope with LAS V2.8.1.

### D. Electrophysiology

Recordings were performed on fibroblasts 33-41 days post-infection. Data were acquired in whole-cell mode using an Axopatch 200B amplifier (Molecular Devices) and sampled at 5 kHz with a 2 kHz low-pass filter. Recording pipette resistance was 2-6 MΩ. Pipette solution used for voltage and current clamp experiments was (in mM): 130 K-gluconate, 7 KCl, 2 NaCl, 1 MgCl₂, 10 HEPES, 0.4 EGTA, 4 ATP-Mg, 0.3 GTP-Tris adjusted to pH 7.3 with KOH and to 303-309 mOsm with sucrose. Bath solution was (in mM): 150 NaCl, 4 KCl, 2 CaCl₂, 2 MgCl₂, 10 HEPES, 10 glucose adjusted to pH 7.3 with NaOH and to 312-318 mOsm with sucrose. In current clamp, cells were initially injected with -300 pA for 100 ms followed by steps from 0 to +800 pA for 1 s in 100 pA increments. In voltage clamp, cells were held at −70 mV and stepped from −70 mV to +70 mV for 200 ms in 10 mV increments. Addition of 1 μM TTX (Tocris Bioscience) was used where indicated. Series resistance was left uncompensated due to the fragility of the cells, but was corrected in the current clamp calculations. The liquid junction potential was calculated to be 15 mV (Clampfit) and corrected in calculating resting membrane potentials as according to previously published methods (Barry, P.H. (1994) JPCaIc, a software package for calculating liquid junction potential corrections in patch-clamp, intracellular, epithelial and bilayer measurements and for correcting junction potential measurements. J Neurosci Methods 51(1):107-116). For glutamate application experiments, the solution applied was Mg2+-free.

### E. Calcium Imaging

Cells were loaded with Fluo-2 AM (5 uM, TEFLABS) in tyrode solution for 30 minutes in 37° C. incubator. After two washes with tyrode, cells were imaged using a filter cube (excitation 470+/−20 nm and Emission 535+/−50 nm). In some cases, 1 μM TTX or 200 μM CdCl2 were perfused. All images were converted to TIFF files and analyzed off-line with Metamorph or ImageJ. All error bars represent SEM. For analysis of FM positive puncta, 1.3 pm in diameter ROI were used to cover functional boutons. Photobleaching was corrected by fitting the pre-stimulation baseline by a linear curve.

### F. FM 1-43 Imaging

Cells were perfused with Tyrode solution (containing 150 mM NaCl, 4 mM KCl, 2 mM CaCl₂, 2 mM MgCl₂, 10 mM glucose, 10 mM Hepes, 310-315 mOsm, with pH at 7.35). Switching of perfusion solution was carried out with a precision of <2 s. Solutions contained 10 pM NBQX and 50 pM D-APV (Tocris Bioscience) to prevent possible recurrent activity and synaptic plasticity. All experiments were performed at room temperature and neurons were stimulated with platinum electrodes. Putative presynaptic boutons were stained with 8 μM FM1-43 (Molecular Probes) using field stimulation for 120 s at 10 Hz, followed by 60 s without stimulation to maximize the loading. In some experiments, 0.1 mM CaCl₂ was used to test the calcium dependency. After 10 min of washing with dye-free Tyrode's solution, individual boutons were destained by field stimulation. Image acquisition was conducted as previously described (Zhang et al., 2009). FM1-43 dyes were excited at 470 nm (D470-40×; Chroma) and their emission was collected at 535 nm (535/50m). TurboRed was excited at 535 nm (535/50ex) and its emission was collected at 580 nm (580 Ip). All images were taken at a frame rate of 1-3 Hz by a Cascade 512B camera.

G. Quantitative Real Time PCR (qRT-PCR)

qRT-PCR was performed using the following primers:

hMAP2:
(SEQ ID NO: 01)
FWD TTCCTCCATTCTCCCTCCTC
(SEQ ID NO: 02)
REV CCTGGGATAGCTAGGGGTTC
hVGLUT1:
(SEQ ID NO: 03)
FWD CGTGAACCACCTGGACATAG
(SEQ ID NO: 04)
REV CCAGGGAGGCAATTAGGAAC
hNMDAR1:
(SEQ ID NO: 05)
FWD AGACGTGGGTTCGGTATCAG
(SEQ ID NO: 06)
REV CATCCTTGTGCCGCTTGTAG
hHPRT:
(SEQ ID NO: 07)
FWD TCCTTGGTCAGGCAGTATAATCC
(SEQ ID NO: 08)
REV: GTCAAGGGCATATCCTACAACAAA

RNA was extracted by RNeasy Plus Micro Kit (Qiagen) and cDNA was prepared using Superscript II (Invitrogen) according to manufacturer's protocols. qRT-PCR for miR-9. miR-9*and miR-124 were performed using TaqMan miR-9, miR-9* and miR-124 microRNA assay kit (Applied Biosystems) using RNA extracted by Trizol (Invitrogen). All of real time PCR was performed in 7500 Fast Real Time PCR System (Applied Biosystems).

Single cell RT-PCR was performed using the following primers:

| | |
|---|---|
| Crygs-f | CAGACTTCCGCTCGTACCTAA (SEQ ID NO: 09) |
| Crygs-r | TCGCCCTGGGGTAAGATGT (SEQ ID NO: 10) |
| hBDNF-F | GATGCTCAGTAGTCAAGTGCC (SEQ ID NO: 11) |
| hBDNF-R | GCCGTTACCCACTCACTAATAC (SEQ ID NO: 12) |
| hBSN-F | CCACATCACCCTACTCCGTC (SEQ ID NO: 13) |
| hBSN-R | TTGCAGACCTTGTTGTGACAC (SEQ ID NO: 14) |
| hCRIM1-F | GCGTTTGCGAAGATGAGAACT (SEQ ID NO: 15) |
| hCRIM1-R | TGGTGTTACATTCACATTTCCCA (SEQ ID NO: 16) |
| hCTIP2-BCL11B-F | TGGGTGCCTGCTATGACAAG (SEQ ID NO: 17) |
| hCTIP2-BCL11B-R | GGCTCGGACACTTTCCTGAG (SEQ ID NO: 18) |
| hCUX1-F | GCTCTCATCGGCCAATCACT (SEQ ID NO: 19) |
| hCUX1-R | TCTATGGCCTGCTCCACGT (SEQ ID NO: 20) |
| hCUX2-f | CGAGACCTCCACACTTCGTG (SEQ ID NO: 21) |
| hCUX2-r | TGTTTTTCCGCCTCATTTCTCTG (SEQ ID NO: 22) |
| hDBH-f3 | CTGAAGCCCAATATCCCCGAA (SEQ ID NO: 23) |
| hDBH-r3 | GTAGCACCAGTACGTGGTCTC (SEQ ID NO: 24) |
| hDDC-F | ACTGGCTCGGGAAGATGCT (SEQ ID NO: 25) |
| hDDC-R | CCGATGGATCACTTTGGTCC (SEQ ID NO: 26) |
| hDKK3-F | TGGGGTCACTGCACCAAAAT (SEQ ID NO: 27) |
| hDKK3-R | GAAGGTCGGCTTGCACACATA (SEQ ID NO: 28) |
| hDLX5-F | AGCTCCTACCACCAGTACGG (SEQ ID NO: 29) |
| hDLX5-R | GTTTGCCATTCACCATTCTCAC (SEQ ID NO: 30) |
| hDSCAM-F | TTTTACGGGAGCCCTATACAGT (SEQ ID NO: 31) |
| hDSCAM-R | GCAACATTGCCTCTCATGGTTT (SEQ ID NO: 32) |
| hETV1-F | CTGGATGACCCGGCAAATTCT (SEQ ID NO: 33) |
| hETV1-R | CCTCTTCAGGCTCAATCAGTTT (SEQ ID NO: 34) |
| hFOXP2-F | TTTCTAAAGAACGCGAACGTCT (SEQ ID NO: 35) |
| hFOXP2-R | GCAATATGCACTTACAGGTTTGG (SEQ ID NO: 36) |
| hGAPDH-f2 | CATGAGAAGTATGACAACAGCCT (SEQ ID NO: 37) |
| hGAPDH-r2 | AGTCCTTCCACGATACCAAAGT (SEQ ID NO: 38) |
| hGRIN1-NR1-f | AGGAACCCCTCGGACAAGTT (SEQ ID NO: 39) |
| hGRIN1-NR1-r | CCGCACTCTCGTAGTTGTG (SEQ ID NO: 40) |
| hGRIN2A-NR2A-f | GGGCTGGGACATGCAGAAT (SEQ ID NO: 41) |
| hGRIN2A-NR2A-r | CGTCTTTGGAACAGTAGAGCAA (SEQ ID NO: 42) |
| hGRIN2B-NR2B-f | GTAGCCATGAATGAGACCGAC (SEQ ID NO: 43) |
| hGRIN2B-NR2B-r | GGATCGGGGTGAGAGTCTGT (SEQ ID NO: 44) |
| hGRIN3A-NR3-f | GACGCCCTCCTATTTGCCG (SEQ ID NO: 45) |
| hGRIN3A-NR3-r | CCACGGTATGGCACACACT (SEQ ID NO: 46) |
| hGRM2-F | TCCTCGACAGTTGCTCCAAG (SEQ ID NO: 47) |
| hGRM2-R | GCAGATGTGGCGTGATCCAT (SEQ ID NO: 48) |
| hGRP-F | GTGGGGCACTTAATGGGGAAA (SEQ ID NO: 49) |
| hGRP-R | CTATGAGACCCAGCAAATTCCTT (SEQ ID NO: 50) |
| hIGFBP4-F | GGTGACCACCCCAACAACAG (SEQ ID NO: 51) |
| hIGFBP4-R | GAATTTTGGCGAAGTGCTTCTG (SEQ ID NO: 52) |
| hLHX2-f | CCTGGTCTACTGCCGCTTG (SEQ ID NO: 53) |
| hLHX2-r | GTTGAAGTGTGCGGGGTACT (SEQ ID NO: 54) |
| hLXN-F | AACGGGACAAGAAACTGCAC (SEQ ID NO: 55) |
| hLXN-R | CTAGCGGTTCCTTCATGGACT (SEQ ID NO: 56) |
| hMAPT-Tau-f | TACAAACCAGTTGACCTGAGCA (SEQ ID NO: 57) |
| hMAPT-Tau-r | ATGGATGTTGCCTAATGAGCC (SEQ ID NO: 58) |
| hMEF2c-f | ATGCCATCAGTGAATCAAAGGAT (SEQ ID NO: 59) |
| hMEF2c-r | CTGGTAAAGTAGGAGTTGCTACG (SEQ ID NO: 60) |
| hMGLUR1-F | CCAGCGATCTTTTTGGAGGTG (SEQ ID NO: 61) |
| hMGLUR1-R | TGGTGATGGACTGAGAAGAGG (SEQ ID NO: 62) |
| hNCAM-F | ACATCACCTGCTACTTCCTGA (SEQ ID NO: 63) |
| hNCAM-R | CTTGGACTCATCTTTCGAGAAGG (SEQ ID NO: 64) |
| hNR4A3-F | CTGAGCATGTGCAACAATTCTAC (SEQ ID NO: 65) |
| hNR4A3-R | ACAGCTCCAAAAAGGCTGATTC (SEQ ID NO: 66) |
| hNSE-F | GGAGTTGGATGGGACTGAGAA (SEQ ID NO: 67) |
| hNSE-R | CTGAGCAATGTGGCGATACAG (SEQ ID NO: 68) |
| hNTF3-F | CAGAGACGCTACAACTCACCG (SEQ ID NO: 69) |
| hNTF3-R | CCGTGATGTTCTGTTCGCC (SEQ ID NO: 70) |
| hNTN1-F | TGCAAGCCCTTCCACTACG (SEQ ID NO: 71) |
| hNTN1-R | TGTTGTGGCGACAGTTGAGG (SEQ ID NO: 72) |
| hNTSR1-F | CTGACGGTGCCTATGCTGTTC (SEQ ID NO: 73) |
| hNTSR1-R | GAAGGTGTTGACCTGTATGACG (SEQ ID NO: 74) |
| hNUPR1-F | CTCTCATCATGCCTATGCCTACT (SEQ ID NO: 75) |
| hNUPR1-R | CCTCCACCTCCTGTAACCAAG (SEQ ID NO: 76) |
| hOCT3/4-F | GGGAGATTGATAACTGGTGTGTT (SEQ ID NO: 77) |
| hOCT3/4-R | GTGTATATCCCAGGGTGATCCTC (SEQ ID NO: 78) |
| hOMA1-F | TAGGCAGGGGCATAAGGAAAT (SEQ ID NO: 79) |
| hOMA1-R | CTCAAACCAAGGAATAGCTTCCA (SEQ ID NO: 80) |
| hPCLO-F | CAGACACTTTCAGGTCAGAGC (SEQ ID NO: 81) |
| hPCLO-R | AGGCATCATACTAGACTTGTGCT (SEQ ID NO: 82) |
| hPCP2-F | AGAGGCCAGCAGAAAAGTGACT (SEQ ID NO: 83) |
| hPCP2-R | GTGGCTCAGCAGATTGAAGAA (SEQ ID NO: 84) |
| hPERIPHERIN-F | CCAAGTACGCGGACCTGTC (SEQ ID NO: 85) |
| hPERIPHERIN-R | CTCGCACGTTAGACTCTGGA (SEQ ID NO: 86) |
| hPLXND1-F | CATGGAGATGGCCTGTGACTA (SEQ ID NO: 87) |
| hPLXND1-R | GGAAGGGCGGAAACTGGTC (SEQ ID NO: 88) |
| hPPP1R1B-DARPP32F | AGTCTGCTGGGCAAAAGACAA (SEQ ID NO: 89) |
| hPPP1R1B-DARPP32R | AGGCTCACTTAGTGCTGGGT (SEQ ID NO: 90) |
| hPSD93-DLG2-F | GGCCTGGGATTCAGTATTGCT (SEQ ID NO: 91) |
| hPSD93-DLG2-R | CCCGCAAGATACAATCATTGACC (SEQ ID NO: 92) |
| hPSD95-DLG4-F | TCACAACCTCTTATTCCCAGCA (SEQ ID NO: 93) |
| hPSD95-DLG4-R | CATGGCTGTGGGGTAGTCG (SEQ ID NO: 94) |
| hRAC3-F | CCGTGGGGAAGACATGCTT (SEQ ID NO: 95) |
| hRAC3-R | ACCATCACGTTGGCAGAGTAG (SEQ ID NO: 96) |
| hSATB2-F | TCTCCCCCTCAGTTATGTGAC (SEQ ID NO: 97) |
| hSATB2-R | AGGCAAGTCTTCCAACTTTGAA (SEQ ID NO: 98) |
| hSCN1A-RD1 | TGGGGAGTGGATAGAGACCA (SEQ ID NO: 99) |
| hSCN1A-RD2 | GAAAGAGATTCAGGACCACTAGG (SEQ ID NO: 100) |
| hSCN2A-RD10 | GGTGATTGGAAATCTAGTGGTTC (SEQ ID NO: 101) |
| hSCN2A-RD11 | CATCCTTCCCACAGCAATCT (SEQ ID NO: 102) |
| hSCN3A-RD16 | AGTAGTGGTGCATTGGCCTT (SEQ ID NO: 103) |
| hSCN3A-RD17 | GCAACCCATTTGAGAAGCAT (SEQ ID NO: 104) |
| hSCN8A-RD19 | ACAGGAAGAGGCACAGGC (SEQ ID NO: 105) |
| hSCN8A-RD20 | CCCCTCCTTCTTCACCTTCT (SEQ ID NO: 106) |
| hSEMA3E-F | ATTGTTTGCTGGACTCTACAGTG (SEQ ID NO: 107) |
| hSEMA3E-R | CTTTCAACAGACGCTCATCGT (SEQ ID NO: 108) |
| hSOMATOSTATIN-F | GCTGCTGTCTGAACCCAAC (SEQ ID NO: 109) |
| hSOMATOSTATIN-R | CGTTCTCGGGGTGCCATAG (SEQ ID NO: 110) |
| hSOX5-F | CAGAGTGGCGAGTCCTTGTC (SEQ ID NO: 111) |
| hSOX5-R | TTTCTTCCGGCTCGTTTTTGA (SEQ ID NO: 112) |
| hSYNAPSIN-1-F | TGAAGCCGGATTTTGTGCTGA (SEQ ID NO: 113) |
| hSYNAPSIN-1-R | GACCAAACTGCGGTAGTCTCC (SEQ ID NO: 114) |
| hSYT9-F | TGGCAGACGACTGAAGAAGAG (SEQ ID NO: 115) |
| hSYT9-R | GGATTTGGTCAATGTTCTCGGG (SEQ ID NO: 116) |
| hTACR3-F | TTCATAGCGAGTGGTACTTTGGC (SEQ ID NO: 117) |
| hTACR3-R | AGTCTGGGTTTCAAGGGATCA (SEQ ID NO: 118) |
| hTBR1-f2 | CATTATCTCGACCACTGACAACC (SEQ ID NO: 119) |
| hTBR1-r2 | AGACCCCGTCCAAGACAGG (SEQ ID NO: 120) |
| hTH-F | GCCCTACCAAGACCAGACGTA (SEQ ID NO: 121) |
| hTH-R | CGTGAGGCATAGCTCCTGA (SEQ ID NO: 122) |
| hTIS21-BTG2-f | CAGAGCACTACAAACACCACTG (SEQ ID NO: 123) |
| hTIS21-BTG2-r | CTGAGTCCGATCTGGCTGG (SEQ ID NO: 124) |
| hTLE1-f | AAGTTCACTATCCCGGAGTCC (SEQ ID NO: 125) |
| hTLE1-r | TCTGTCTTTTCACTTGCCAGTTT (SEQ ID NO: 126) |
| hTLE4-F | ACAAGCAGGCAGAGATTGTCA (SEQ ID NO: 127) |
| hTLE4-R | TCCATGTGATAAATGCTGGGC (SEQ ID NO: 128) |
| hTPM2-F | CTGAGACCCGAGCAGAGTTTG (SEQ ID NO: 129) |
| hTPM2-R | TGAATCTCGACGTTCTCCTCC (SEQ ID NO: 130) |
| hu-GRM1-f | AGACCAATGAGACGGCCTG (SEQ ID NO: 131) |
| hu-GRM1-r | CCTCCTCTACGTTGTAAAGGGT (SEQ ID NO: 132) |
| hu-GRM5-f | TCCAATCTCCCGATGTCAAGT (SEQ ID NO: 133) |
| hu-GRM5-r | TCGGCACTGAAAACGATGCT (SEQ ID NO: 134) |

### II. RESULTS AND DISCUSSION

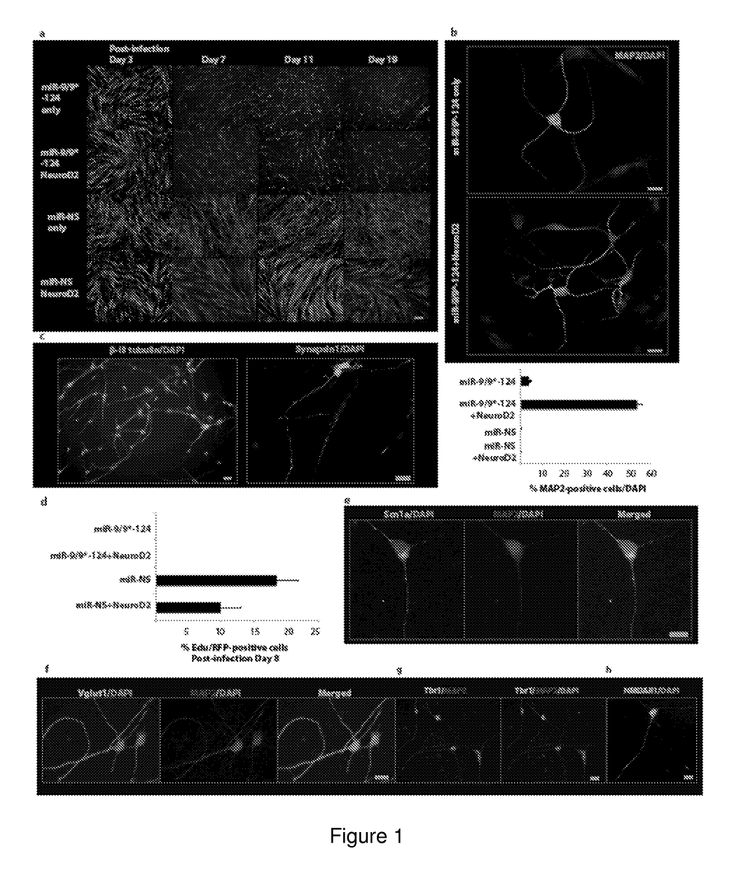

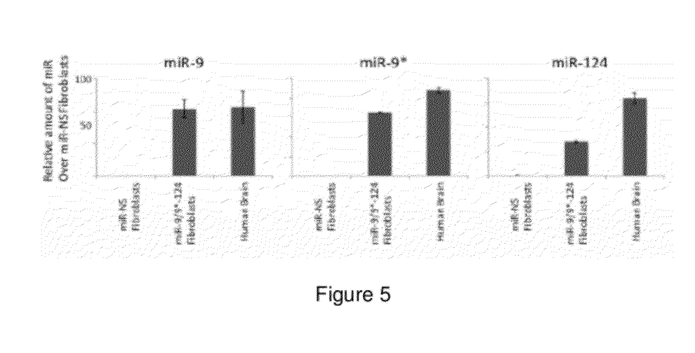

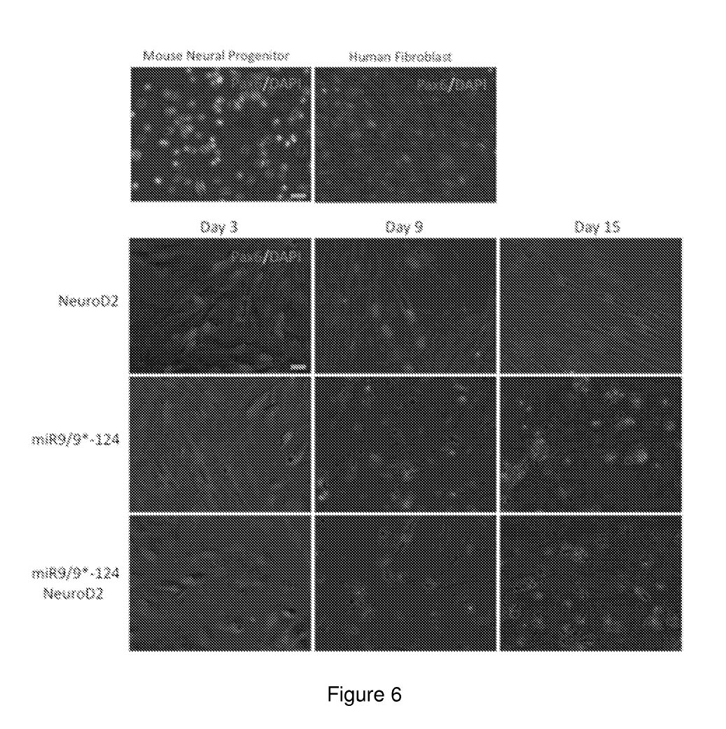

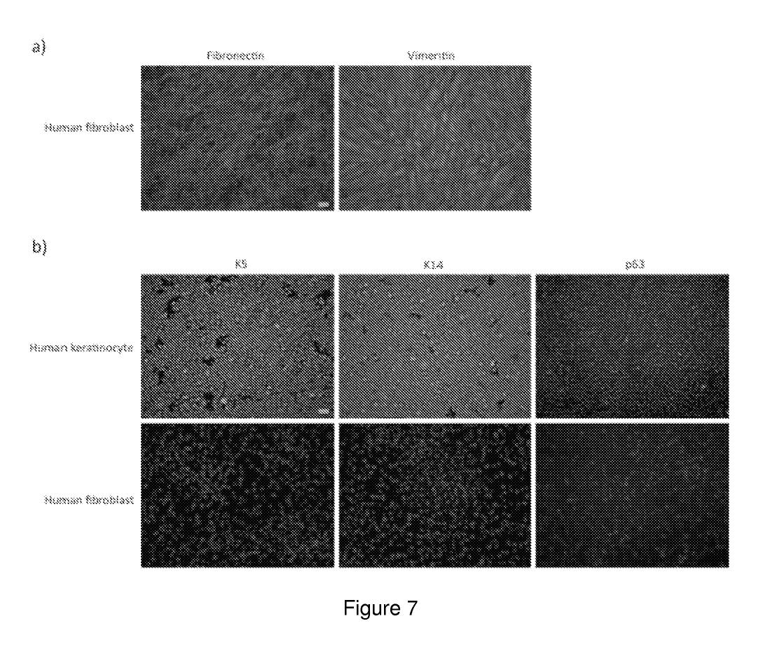

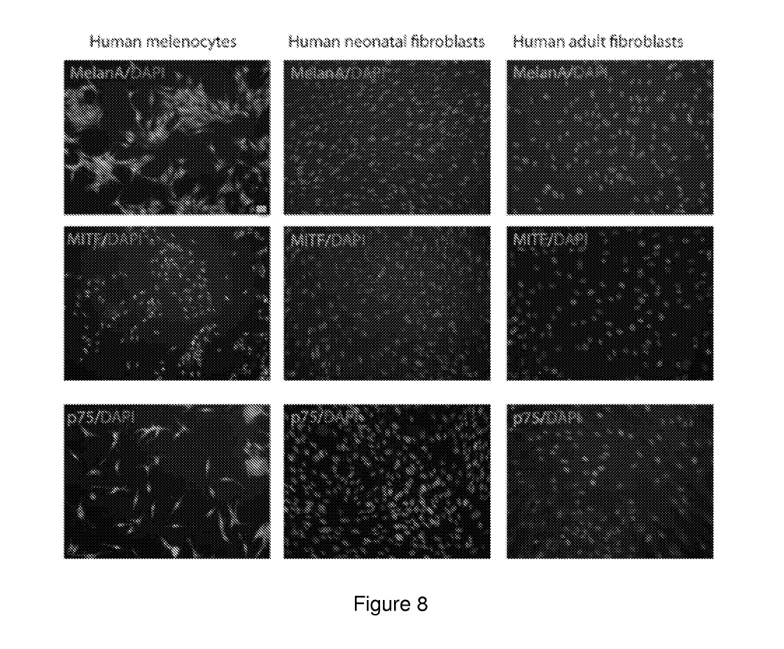

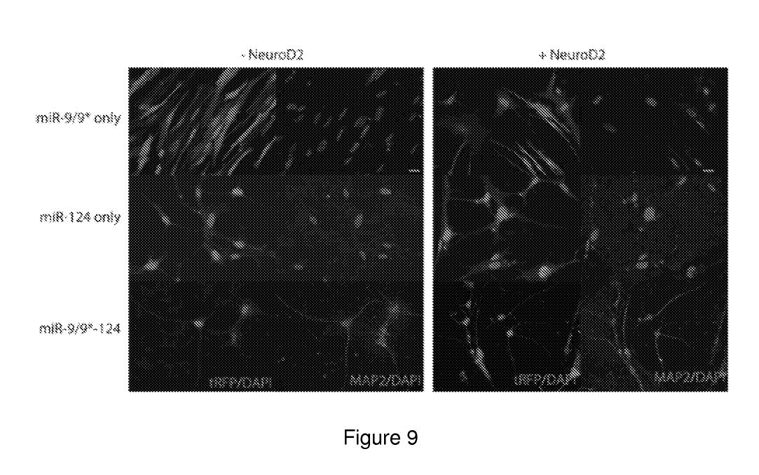

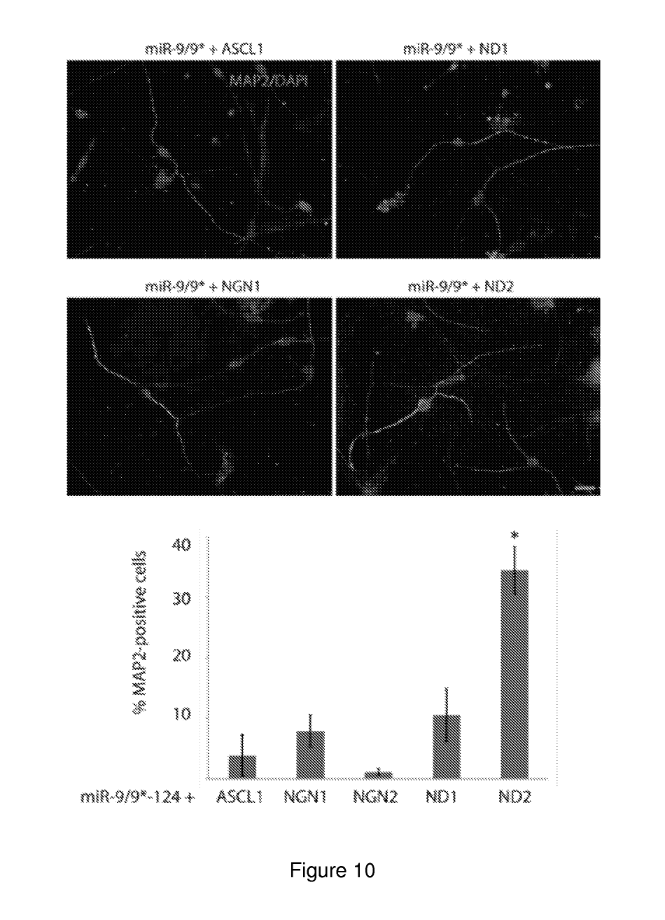

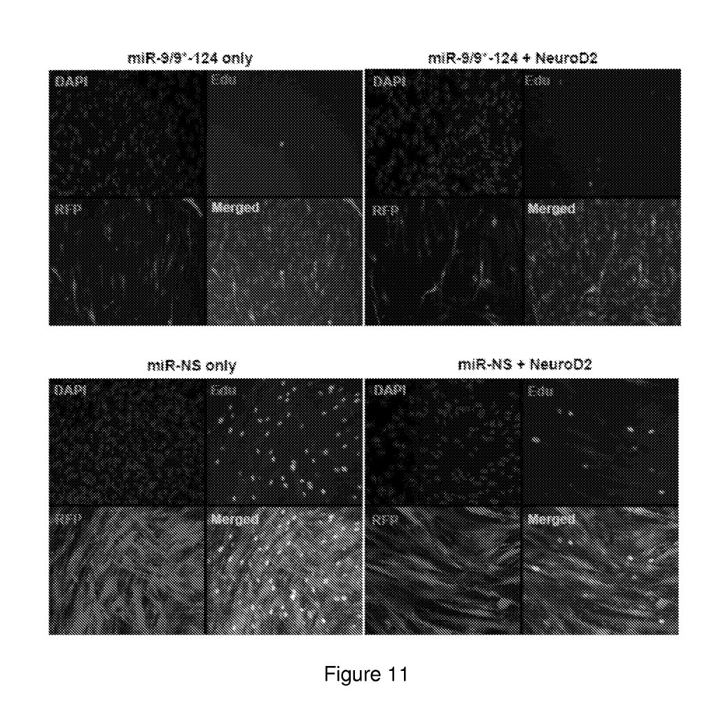

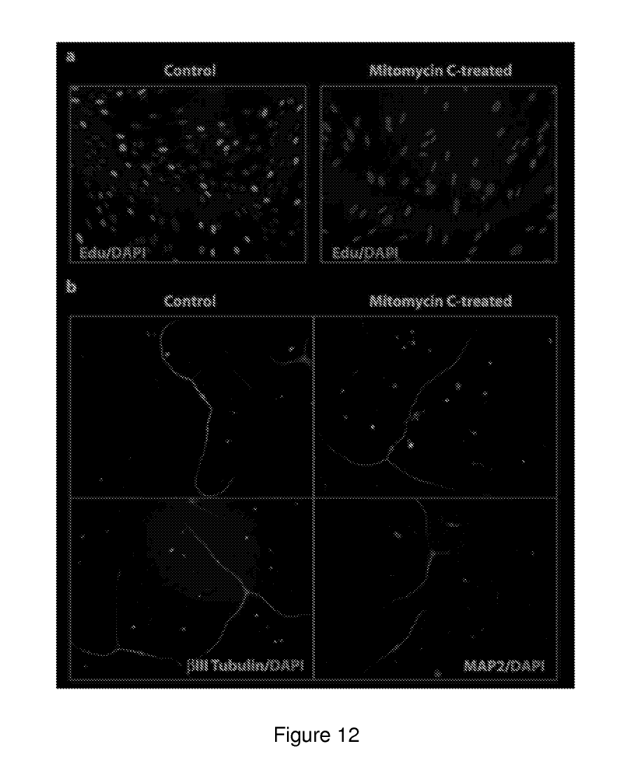

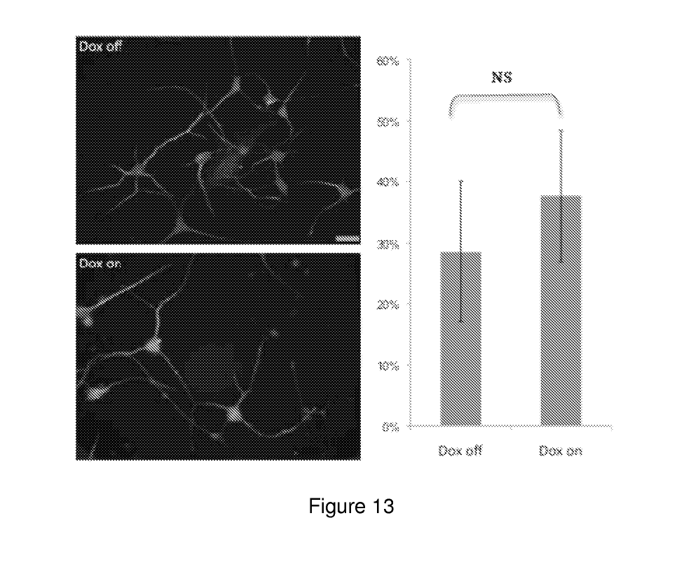

We tested if miR-9/9* and miR-124 could direct reprogramming of cell fates towards neurons if they were relieved of their normal repression by REST(Conaco, C., Otto, S., Han, J. J., & Mandel, G., Reciprocal actions of REST and a microRNA promote neuronal identity. Proc Natl Acad Sci U S A 103 (7), 2422-2427 (2006)) in fibroblasts. We prepared a single lentiviral vector that expresses both precursors of miR-9/9* and miR-124 (miR-9/9*-124) along with a turbo red fluorescent protein (tRFP) marker and infected human neonatal foreskin fibroblasts (FIG. 1 and FIG. 5). This starting culture of fibroblasts was free of any neural progenitors (as monitored by the expression of PAX6, SOX2 and Tbr2, FIG. 6), =keratinocytes (FIG. 7), and melanocytes (FIG. 8), indicating that cells of ectodermal origin were not present in our fibroblast cultures. Remarkably, the dermal fibroblasts expressing the microRNAs showed a rapid reduction in proliferation and neuron-like morphologies within two weeks (FIG. 1a). We found that the infected cells began to express MAP2, a marker of post-mitotic neurons within 4 weeks post-infection (FIG. 1b, top panel). This was due to synergism between miR-9/9* and miR-124 since expressing these microRNAs separately did not lead to appearance of MAP2-positive cells (FIG. 9). Due to the low percentage of MAP2-positive cells using microRNAs only (less than 5% of the cells counted), we tested several neurogenic transcription factors involved in neural differentiation to enhance cell fate conversions. We focused on neurogenic factors belonging to the basic helix-loop-helix group, including Neurogenin1, Neurogenin2, ASCL1, NeuroD1 and NeuroD2, and found that NeuroD2, a neurogenic factor required for proper neural development, was most effective at enhancing the frequency of conversion to cells with neural characteristics (FIG. 1b and FIG. 10). By counting the number of MAP2-positive cells remaining by the end of 30 days post-infection, we estimated that approximately 50% of the remaining cells appeared to have acquired neuronal fates (FIG. 1b, graph). However, because cells detach, are uninfected or die during the course of conversion, a conservative estimate is that -5% of the initial cells became neurons. Importantly, expression of NeuroD2 alone could not accomplish this conversion by itself, and non-specific microRNA was also ineffective (FIG. 1b, graph), demonstrating the essential role of miR-9/9*-124 in inducing neuronal conversion of fibroblasts. Furthermore, expressing miR-9/9* and miR-124 individually with NeuroD2 did not lead to appearance of MAP2-positive cells (FIG. 9), again demonstrating the importance of synergism between miR-9/9* and miR-124 for the conversion. The infected cells expressed several neuron-specific markers including the neuron-specific β-III tubulin and Synapsinl (FIG. 1c), suggesting that in addition to morphological conversion, the infected fibroblasts were activating neuronal genes. By assessing EdU-incorporation, we found that miR-9/9*-124 had anti-proliferation effect on infected fibroblasts, and by the end of the first week post-infection, most of the RFP-positive cells appeared to have exited cell cycle (FIG. 1d and FIG. 11). Inhibiting the proliferation of starting fibroblast culture with mitomycin C did not interfere significantly with the conversion process, indicating that the conversion occurs directly from the fibroblasts (FIG. 12). As the expression of microRNAs appears to be the major determinant of the cell fate changes, we asked whether the converted cells would revert to fibroblasts if miR-9/9*-124 expression is interrupted. We used a doxycycline-responsive promoter to express miR-9/9*-124, along with NeuroD2 and removed the activator after 30 days post-infection. As indicated by the continuous expression of MAP2 7 days after the removal of doxycycline, the conversion appeared to be stable for the times tested (FIG. 13). We observed some degree of reversion back to fibroblasts when doxycycline was removed in the first 3 weeks post-infection (data not shown), thus we estimated the critical time for the conversion to be within 3-4 weeks post-infection. After 4 weeks, the cells appeared to be stable with no detectable rate of reversion for up to three months, despite the inactivation of the transgenes (as evidenced by loss of RFP) in many cells. These findings shows that transient expression of miR-9/9*- 124 (3-4 weeks) leads to a stable neuronal genetic circuit.

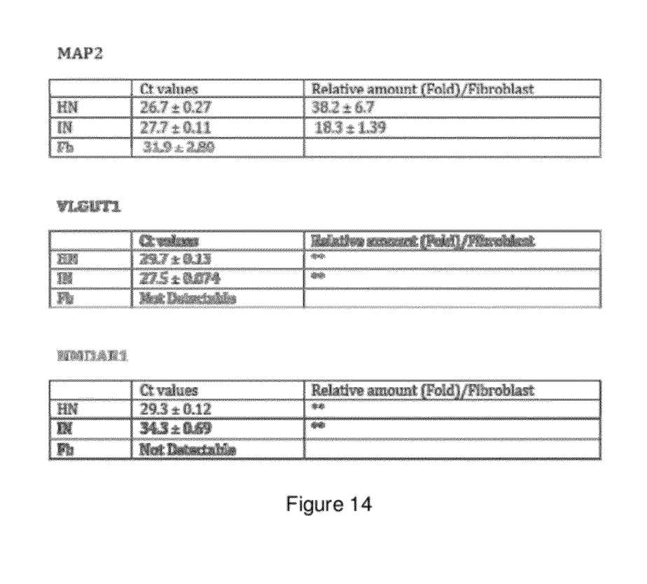

Because of the higher efficiency of transformation with NeuroD2 compared to other neurogenic factors, we decided to focus on characterizing the cells overexpressing miR-9/9*-124 and NeuroD2. The converted cells express sodium channels as assayed by immunostaining using antibodies against SCN1a, alpha subunit of voltage-gated sodium channels (FIG. 1e), an essential feature of excitability of neurons. When we analyzed expression of proteins that are characteristic for different types of neurons, we found that nearly all MAP2-positive cells derived from the conversion coexpressed the vesicular glutamate transporter, VGLUT1, indicating that the converted cells adopted traits of glutamatergic neurons (FIG. 1f, refer to FIG. 14 for quantitative real time PCR data). We could not detect the expression of the vesicular GABA (γ-aminobutyric acid) transporter VGAT, a marker of GABAergic inhibitory neurons. In addition, we could not detect the expression of markers of other types of neurons including tyrosine hydroxlyase, choline acetyltransferase and serotonin for doparminergic, cholinergic and serotonergic neurons, respectively. Peripherin (a marker of neurons of peripheral nervous system) and Islet2 (a marker of ventral motor neurons) were also absent in the transformed cells. Interestingly, the majority of MAP2-positive cells (99 out of 106 cells) expressed TBR1, a marker of excitatory cortical neurons (FIG. 1f) and expressed glutamate-gated ion channels, including the R1 subunit of NMDA receptors (FIG. 1g). These results indicate that the converted cells are excitatory glutamatergic neurons characteristic of the cerebral cortex.

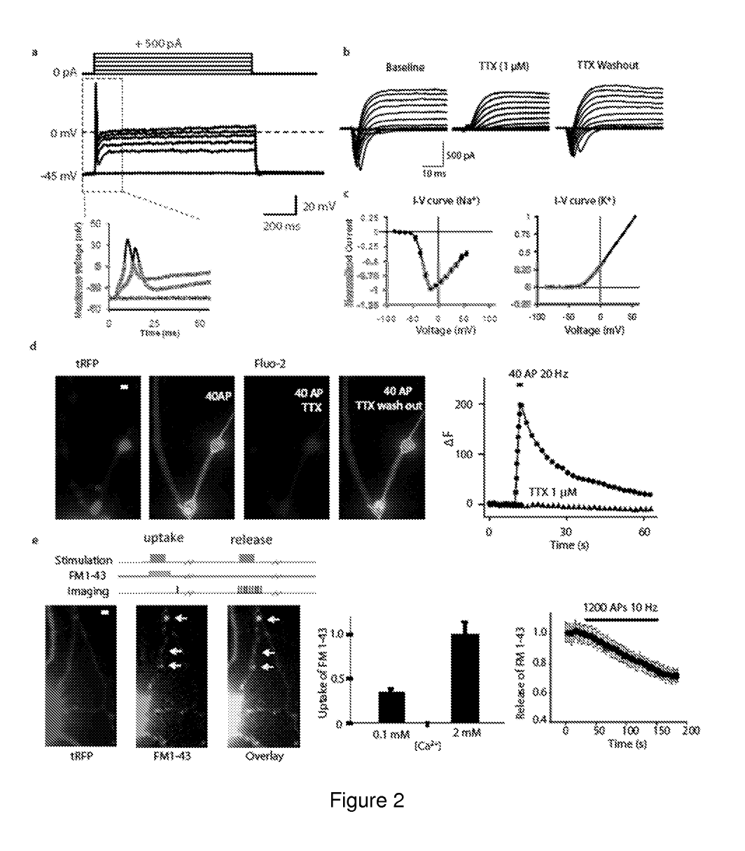

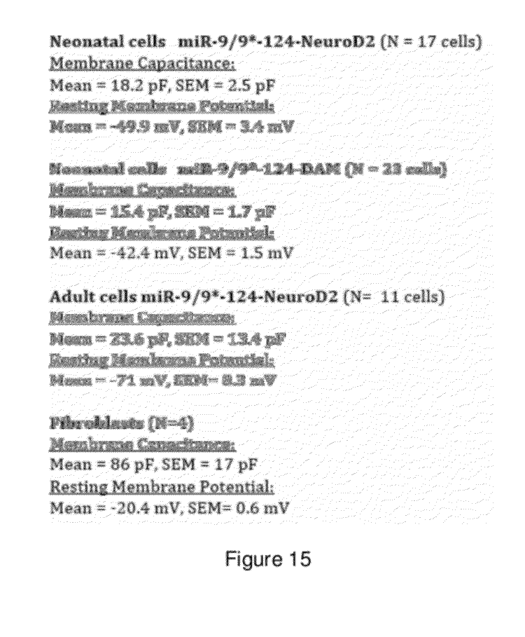

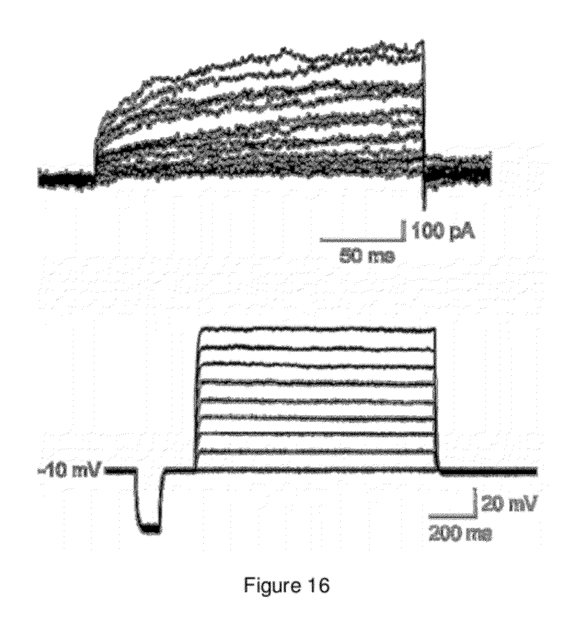

We used whole cell patch clamping to analyze the electrophysiological properties of the converted cells. Whereas control fibroblasts showed no apparent depolarization-dependent inward current (FIG. 16), injecting depolarizing current in induced neurons (cultured up to 2 months) could consistently trigger a single spike of action potential (FIG. 2a). . Moreover, the converted cells showed resting membrane potentials (−49.9±3.4 mV, N=17) whereas control fibroblasts showed significantly less negative resting membrane potentials (−20.4±0.6 mV, N=4) (FIG. 15). We then employed voltage clamp to analyze the functional properties of ion channels in the converted cells. Large inward currents closely followed by outward currents were observed when a series of voltage steps were applied in the induced cells (FIG. 2a) whereas control fibroblasts failed to do so (FIG. 16). Importantly, the addition of 1 pM TTX completely and reversibly blocked the initial inward current, confirming that the current is indeed due to bona fide voltage-gated sodium channels (FIG. 2b), as would be expected from the I-V curve of sodium currents (FIG. 2c, left). The converted cells also displayed robust voltage-dependent outward currents typical of those found in neurons (FIG. 2c, right). Together, these data show that miR-9/9*-124- NeuroD2-converted cells display electrophysiological properties of neurons

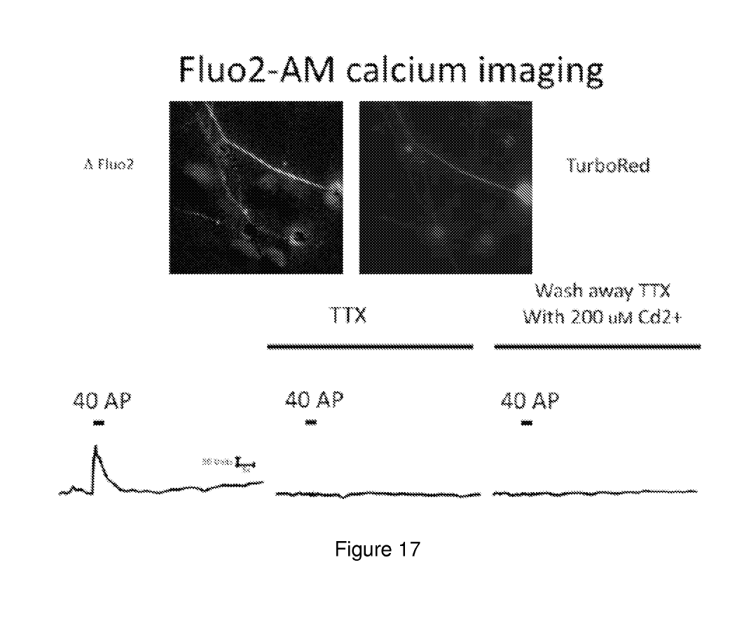

Another critical aspect of neuronal identity is the ability to form functional synapses in which action potentials trigger calcium-dependent neurotransmitter release. We analyzed the converted cells' ability to elicit calcium influx upon stimulation using a Fluo2 indicator. Field stimulation triggered calcium influx that could be abolished by addition of TTX (FIG. 2d) or 200 μM (FIG. 17), indicating the calcium influx was due to activation of voltage-gated calcium channel following action potentials. We used activity-dependent uptake and release of lipophilic dye FM1-43 as a way to evaluate the ability of the induced human neurons to form functional presynaptic terminals (Ryan, T. A. et al., The kinetics of synaptic vesicle recycling measured at single presynaptic boutons. Neuron 11 (4), 713-724 (1993)). We found that the induced cells were able to uptake and release FM dyes in a stimulation-dependent manner (FIG. 2e, top graph). In addition, the uptake of FM dye was Ca²⁺-dependent, as would be expected for neurons (FIG. 2e, bottom graph). Collectively, these results indicate that the induced cells have the electrophysiological excitability and synaptic properties of functional neurons.

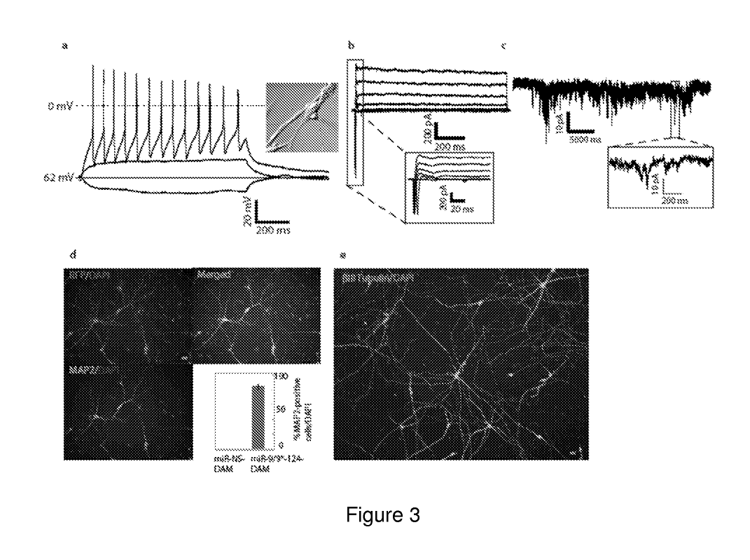

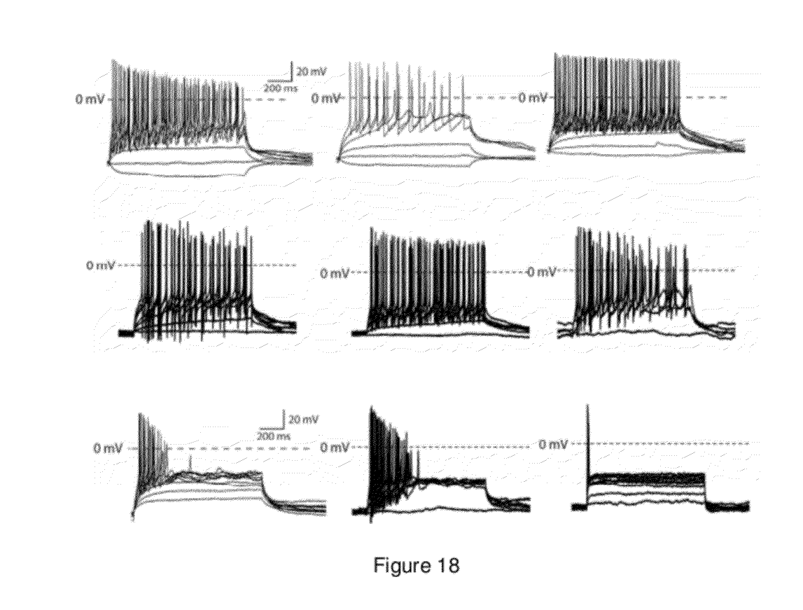

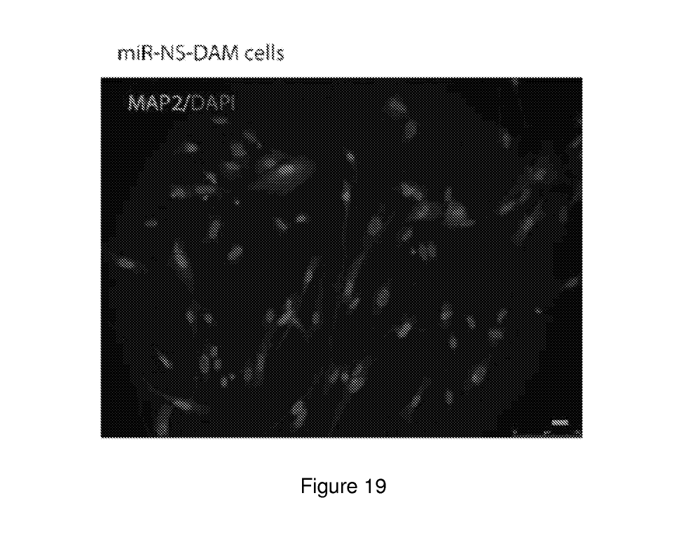

Because the majority of the miR-9/9*-124-NeuroD2-induced cells cultured up to 8 weeks exhibited single action potentials, as commonly observed in immature neurons24, we explored the possibility of accelerating cell maturation by introducing additional neurogenic factors and assaying for the presence of repetitive firing of action potentials.We expressed miR-9/9*-124 together with NeuroD2, ASCL1 and Myt1l (DAM) and found that these converted cells are able to fire repetitive action potentials in response to a single step current injection (FIG. 3a, 19 out of 24 cells randomly selected, FIG. 18) within 5 weeks. The miR-9/9*-124-DAM-induced cells also displayed voltage-dependent sodium inward currents (FIG. 3b), spontaneous synaptic currents (FIG. 3c) and hyperpolarized resting membrane potentials (FIG. 15). Additionally, the miR-9/9*-124-DAMconverted cells were positive for MAP2 expression in approximately 80% of the cells remaining on the coverslips (FIG. 3d), and displayed extensive neurite outgrowth, as illustrated by βIII-tubulin staining (FIG. 3e). Importantly, DAM factors with a nonspecific microRNA failed to produce any neurons as assayed by MAP2 staining (FIG. 19). Thus, we conclude that miR-9/9* and -124 are central components for the neuronal cell fate conversion, and that the neurogenic factors, NeuroD2, ASCL1, and Mtyl1 work synergistically with the microRNAs to induce functional, mature neurons.

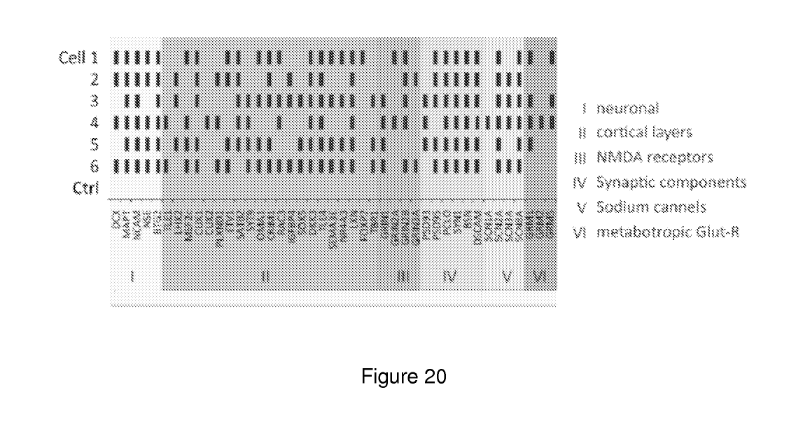

To characterize the identity of the miR-9/9*-124-DAM-converted cells, we removed the cytoplasm of six cells after electrophysiologic analysis using the recording pipette and performed single cell real time RT-PCR using the 96.96 dynamic arrays (Fluidigm) with primer pairs targeted to various genes specific to neuronal subtypes or brain regions. The majority of the cells tested expressed all of five neuronal markers (DCX, MAPT, NCAM, NSE, BTG2, FIG. 20). These cells also expressed NMDA receptor genes (GRIN1, GRIN2A, GRIN2B, GRINS), genes encoding synaptic components (SYN1, PSD93, PSD95, POLO, BSN, DSCAM), sodium channel subunits (SCN1A, SCN2A, SCN3A, SCN8A) and metabotropic glutamate receptors (GRM1, GRM2, GRM5). Furthermore, the induced cells expressed genes specifically expressed in cortical layers including TLE1, LHX2, MEF2c, CUX1, CUX2, PLXND1, ETV1, SATB2, SDYT9, OMA1, CRIM1, RAC3, IGFBP4, SOX5, DKK3, TLE4, SEMA3E, NR4A3, LXN, FOXP2, TBR1 (FIG. 20). In contrast, we did not detect the peripheral nervous system marker (Peripherin), dopaminergic/norepinephric markers (DDC,TH, DBH), striatal markers (DLX5, CTIP2 and DARPP32), or cerebellar genes (PCP2, GRP, TPM2, CRYGS, data not shown). These data show that the miR-9/9*-124- DAM-induced neurons are functional mature neurons with cortical identity.

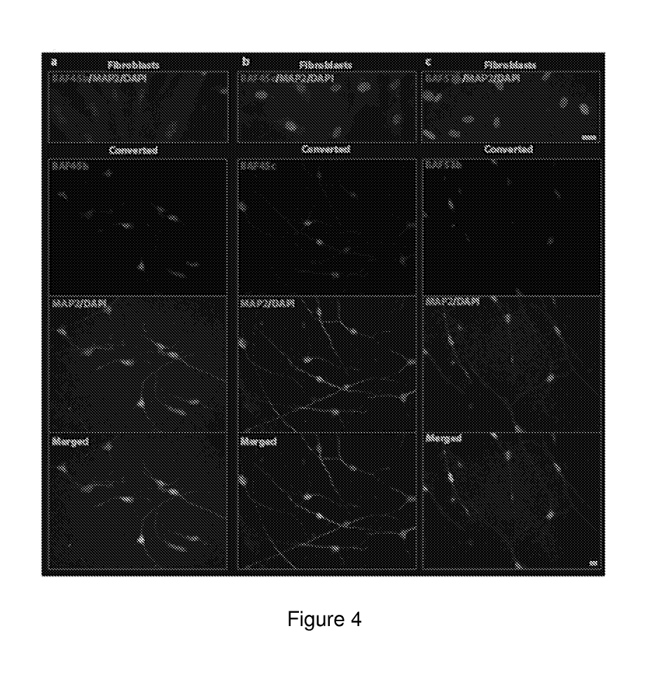

miR-9* and miR-124 are part of a triple negative genetic circuit involving REST repression of miR9* and -124, which in turn repress BAF53a, an actin-related subunit of the SWI/SNF-like npBAF complex found in neural progenitors and other cell types including fibroblasts. Near the time of mitotic exit, BAF53a is repressed by miR-9* and miR-124, a process required to express the neuron-specific BAF53b protein. Mitotic exit is also accompanied by the expression of other neuron-specific subunits, BAF45b and BAF45c, which are components of neural specific nBAF complexes. Since nBAF complexes are essential for neuronal functions, we examined the expression of the subunits of neuron-specific nBAF complexes in the converted neurons. Indeed, the induced neurons expressed each component of nBAF complexes: BAF45b, BAF45c and BAF53b (FIG. 4a-c), demonstrating the assembly of neuron-specific nBAF complexes in the converted cells.

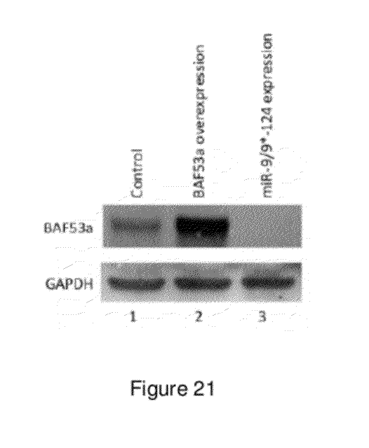

Although this chromatin switch is essential for neural development, there are additional targets of miR-9/9* and miR-124 that also make essential contribution to neurogenesis and the function of neurons. These include components of the REST complex; PTBP-1 whose repression is necessary for the function of PTBP-2, a neuron-specific splicing factor; and TLX, a regulator of neural stem cell self-renewal. We found that human fibroblasts expressed BAF53a, which could be repressed by miR-9/9*-124 (FIG. 21). However, prolonging the expression of BAF53a during the miR-9/9*-124 and NeuroD2-mediated conversion only incompletely blocked neuronal conversion of fibroblasts, as assayed by MAP2 immunostaining (data not shown). Similar results were obtained by extending the expression of REST, CoREST or PTBP1 (data not shown), suggesting that these microRNAs are operating programmatically rather than on a single target in inducing cell fate transformations. These results are consistent with the observation that removing the miR-9* and miR-124 sites in 3′ untranslated region of BAF53a in mice prolongs its expression into the post-mitotic period, but still allows the production of neurons, albeit at a reduced number.

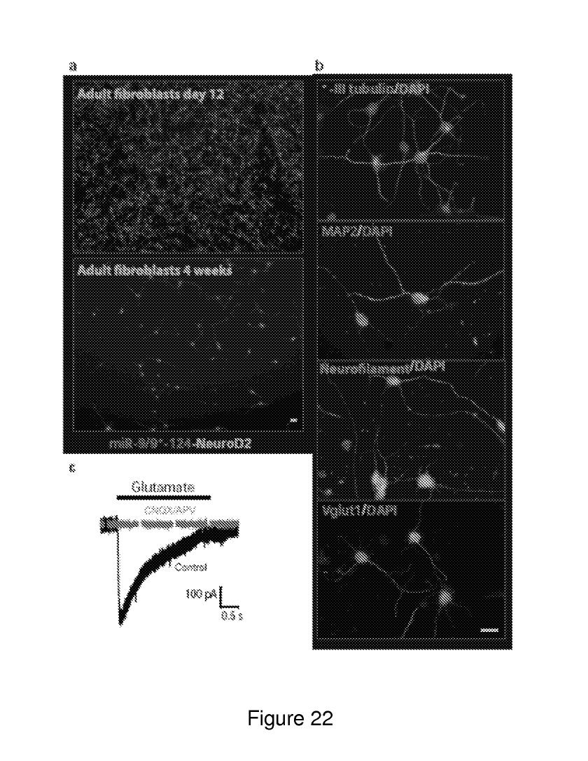

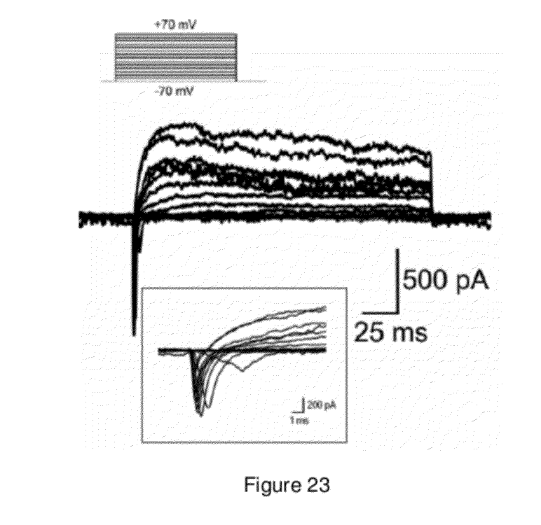

We initiated the neural transformation experiments with neonatal foreskin fibroblasts obtained from American Tissue Culture Collection (ATCC), which we chose because they have been well-characterized by transcription arrays and other studies. We also repeated this result with cultured primary neonatal fibroblasts from a surgical sample. However, for therapeutic purposes the conversion of adult human fibroblasts would be most advantageous since embryonic or neonatal fibroblasts would probably not be available. Hence, we determined if our approach would be effective in transforming adult fibroblasts. We infected human dermal fibroblasts derived from 30 to 45 year-old adults with miR-9/9*-124 and NeuroD2, and monitored morphological changes. We found that the induction was slower for adult fibroblasts compared to neonatal fibroblasts. At day 12 post-infection, we found that many cells still retained the morphology of fibroblasts (-FIG. 22a, top panel) whereas the majority of neonatal fibroblasts had already adopted neuronal morphologies. Nevertheless, we found that by the end of 4 weeks (FIG. 22a, bottom panel), the majority of adult fibroblasts were converted into cells expressing neural specific markers, including β-III tubulin, MAP2, Neurofilament and VGLUT1 (FIG. 22b). We estimated that approximately 32.9±2.7% of the remaining cells counted (±S.E.M., N=94 cells) were converted to MAP2-positive cells (corresponding to about 3-5% of cells initially plated). We also characterized the electrophysiological properties of adult fibroblast-derived neurons. Bath application of glutamate triggered large inward currents that were blocked by a combination of AMPA and NMDA receptor antagonists (Supplementary FIG. 18c). The induced cells had voltage-dependent sodium and potassium conductances (FIG. 23). The converted cells also showed drastically more negative resting membrane potentials (−71±8.3 mV; N=11 cells) than fibroblasts (FIG. 15). We did not observe any conversion using microRNAs only, at least up to 40 day post-infection (data not shown), possibly owing to the slower rate of transformation in adult human fibroblasts. Collectively, these results indicate that miR-9/9*-124 with neurogenic factors can transform adult human fibroblasts into neurons.

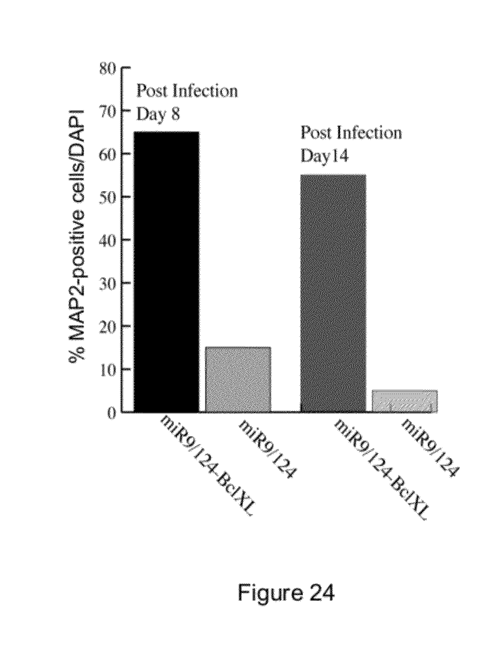

We find that expressing BclXL, an anti-apoptotic member of the BCL-2 protein family, dramatically reduces cell death during the conversion and allows more efficient conversion of human fibroblasts to neurons (FIG. 24; for MiR9/124-NeuroD2, MiR9/124-DAM and miR9/124-BclXL: about 20%, about 35% and about 65% of fibroblasts are directly converted to neurons, respectively.). Therefore, employing a factor such as BclXL permits for the first time the production of quantities of genetically identical neurons necessary for transplantation therapy and also enables biochemical studies on the induced neurons that was not possible in the past.

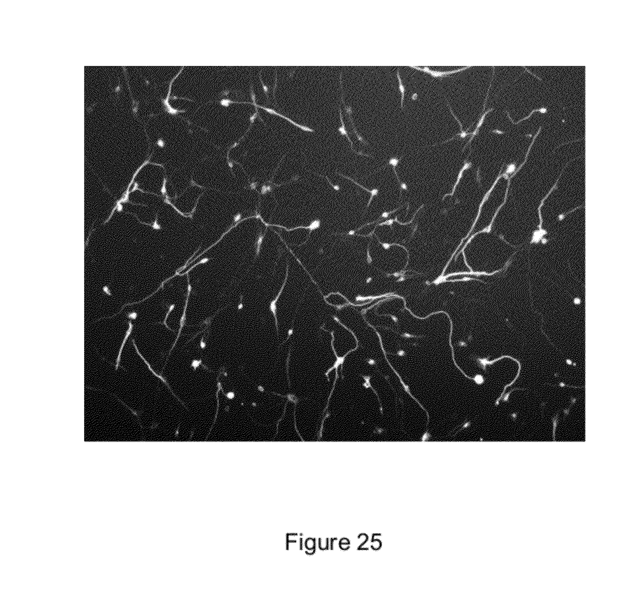

Moreover, we have found an effective route to convert glial cells (non-fibroblast cells) to neurons by using miR9/124 in combination with BclXL as described above (FIG. 25). This method leads to the production of cultures of neurons that are about 35% MAP2-positive with an overall efficiency of about 20%. Because the human brain contains many glial cells, but not fibroblasts, situated near neurons, the ability to produce human neurons from glia allows one to produce neurons in vivo to replace those that are lost through a variety of disease mechanisms. In addition, the ability to convert glia cells can be combined with the use of subtype specific transcription factors to allow in vivo production of therapeutic types of neurons. For example, using methods described herein one can produce Dopaminergic neurons from from local glia allowing the treatment of diseases such a Parkinson's disease. Our studies indicate that the recapitulation of a genetic regulatory circuit involving microRNAs during neural development in human fibroblasts and glia can surprisingly lead to their conversion to neurons. The observation that this transformation can be achieved by experimental alleviation of REST-repression of miR-9/9-124 in human fibroblasts and glia reveals an apparent instructive role for this circuitry. In our study, the role of neurogenic factors, either singly (NeuroD2), or in combination (NeuroD2, ASCL1 and Mtyl1), appears to function synergistically with the neurogenic activities of miR-9/9*-124, One critical role of microRNAs during the conversion probably involves rapid exit from the cell cycle observed in early time points, perhaps due to the repression of BAF53a and other cell cycle regulators. This cell cycle exit is accompanied by the initiation of a program of neuron-specific gene expression allowing the adoption of neuronal fates during the later time course. Previous studies have shown that miR-124 overexpression is not sufficient for conversion of progenitors to neurons (Cao, X., Pfaff, S. L., & Gage, F. H., A functional study of miR-124 in the developing neural tube. Genes Dev 21 (5), 531-536 (2007)). This is consistent with our finding that both miR-9/9* and -124 are both required for the conversion of fibroblasts and glia to neurons. Thus, the mechanism underlying the switching of cell fates probably relies on the synergistic actions of combinations of microRNAs, either through common targets such as BAF53a and/or separate target genes (perhaps additional chromatin-controlling genes).

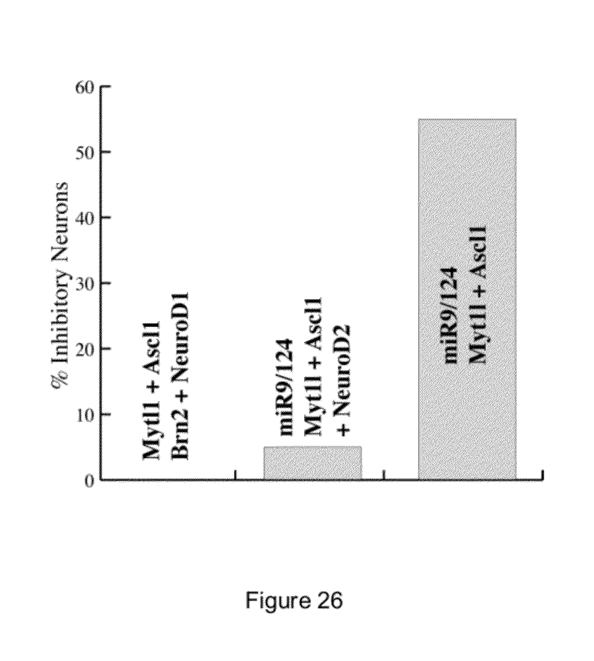

Most miR-9/9*-124-NeuroD2 or DAM-induced neurons have characteristics of excitatory forebrain, cortical neurons. The use of transcription factors other than NeuroD2 in combination with miR-9/9* and miR-124 may be employed for the production of other classes of neurons that could be tailored for specific therapeutic purposes, tissue culture modeling of neurological diseases or drug testing. We find that the use of miR9*, miR124, Ascl and Mytl1 produces populations of neurons about 50% of which appear to be inhibitory neurons, as determined by anti-GABA antibody staining (FIG. 26). This finding is consistent with reports that NeuroD2 opposes the actions of Ascl1 in the generation of gabaergic neurons, see e.g. Roybon, L. et al. (2010) Cereb. Cortex. 20, 1234-44. Since many human diseases are thought to affect inhibitory neurons, authentic disease models can be constructed from the fibroblasts of individuals with specific neurologic diseases. At a practical level, our combined studies provide a relatively simple, yet effective method for converting human fibroblasts and glial cells to neurons, including both excitatory and inhibitory neurons, opening a new platform for studying and treating human neurological diseases.

### III. CONCLUSION

The above discussion demonstrates that expression of miR-9/9*-124 converts human fibroblasts and glial cells to neurons (excitatory and/or inhibitory), an activity that is facilitated by either a neurogenic factor, e.g. NeuroD2, or an agent to block cell death, e.g. BclXL. The two-vector system reported above for conversion of fibroblasts and glial cells to neurons requires about 3-4 weeks and results in cell populations that are approximately 20-65% neurons. These studies show that the genetic circuitry involving miR-9/9*-124 and the nBAF subunits can play an instructive role in neural fate determination. Furthermore, the simplicity and effectiveness of the approach demonstrates that it is a useful tool for studying the pathologenesis of human neurologic diseases.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method of converting a non-neuronal somatic cell into an induced neuronal cell, the method comprising:
contacting the non-neuronal somatic cell with a microRNA mediated neuronal cell induction agent sufficient to cause microRNA mediated conversion of the non-neuronal somatic cell into an induced neuronal cell.

2. The method according to claim 1, wherein the microRNA mediation conversion comprises providing a level of first and second microRNAs in the cell that is sufficient to cause the cell to convert to an induced neuronal cell.

3. The method according to claim 2, wherein the first microRNA is selected from the group consisting of miR-9* and miR-9 and combinations thereof.

4. The method according to claim 3, wherein the second microRNA is miR-124.

5. The method according to claim 4, wherein the method further comprises providing a neurogenic factor activity in the cell.

6. The method according to claim 5, wherein the neurogenic factor is a transcription factor.

7. The method according to claim 6, wherein the transcription factor is selected from the group consisting of: NeuroD2, Myt1l and Ascl1 and combinations thereof.

8. The method according to claim 1, wherein the method further comprises contacting the non-neuronal somatic cell with a conversion enhancement agent.

9. The method according to claim 8, wherein the conversion enhancement agent is an anti-apoptotic agent.

10. The method according to claim 9, wherein the anti-apoptotic agent is BclXL or a nucleic acid encoding the same.

11. The method according to claim 1, wherein the agent is a vector that comprises an expression cassette for at least one microRNA.

12. The method according to claim 1, wherein the agent is an expression inducer.

13. The method according to claim 1, wherein the non-neuronal somatic cell is a vertebrate cell.

14. The method according to claim 13, wherein the vertebrate cell is a mammalian cell.

15. The method according to claim 14, wherein the mammalian cell is a human cell.

16. The method according to claim 15, wherein the human cell is an adult human cell.

17. The method according to claim 16, wherein the adult human cell is a fibroblast cell.

18. The method according to claim 16, wherein the adult human cell is a glial cell.

19. The method according to claim 1, wherein the cell is a member of a population of cells that are collectively contacted with the agent.

20. The method according to claim 1, wherein the induced neuronal cell is an inhibitory neuron.

21. A cell culture system comprising:
non-neuronal somatic cells; and
a microRNA mediated neuronal cell induction agent.

22-26. (canceled)

27. A method of screening candidate agents for neuronal cell induction modulatory activity, the method comprising:
contacting the cell culture system according to claim 21 with a candidate agent; and
comparing the characteristics of the candidate-agent contacted cell culture system with a cell culture system that has not been contacted with the candidate agent to determine whether the candidate agent has neuronal cell induction modulatory activity.

28-42. (canceled)
